(19) United States
(12) Patent Application Publication
(10) Pub. No.: US 2023/0175054 A1
(43) Pub. Date: Jun. 8, 2023

(54) LASH METHODS FOR SINGLE MOLECULE SEQUENCING & TARGET NUCLEIC ACID DETECTION

(71) Applicant: SARMAL, INC., San Diego, CA (US)

(72) Inventor: Inanc ORTAC, San Diego, CA (US)

(21) Appl. No.: 17/915,088

(22) PCT Filed: Mar. 29, 2021
(86) PCT No.: PCT/US2021/024721

§ 371 Date: Sep. 27, 2022

Related U.S. Application Data
(60) Provisional application No. 63/000,979, filed on Mar. 27, 2020.

(51) Int. Cl. C12Q 1/6869

(52) U.S. Cl. CPC C12Q 1/6869

(57) ABSTRACT

Provided herein are methods and systems for sequencing or detecting a single nucleic acid molecule utilizing components for a luminescence reaction.

### TECHNICAL FIELD

The invention relates to methods for single molecule nucleic acid sequencing and detection of a target sequence.

### INTRODUCTION

Current sequencing technologies can be grouped into two main categories: short-read sequencing and long-read sequencing. In each category, DNA is cleaved into pieces with lengths up to a certain number of nucleotides or basepairs (bp). In all cases, all pieces of DNA are spread into a 2 dimensional array and are detected by a sensor array corresponding to where at least one sensor is matched with a piece of DNA.

Short-read sequencing approaches are simple cycle based technologies that includes sequencing-by-ligation (SBL) and sequencing-by-synthesis (SBS). SBL approaches includes SOLID (Thermo Fisher) and Complete Genomics (BGI). With SOLID, read lengths around 75 basepairs (bps) are reached while with the Complete Genomics approach, 28 to 100 basepair reads are feasible. With these approaches structural variation and genome assembly are not possible and they are susceptible to homopolymer errors. Their runtimes are on the order of several days. Illumina and Qiagen’s GeneReader technology use an SBS approach with Cyclic Reversible Termination. They can reach up to 300 bp. However, a major drawback is under representation of AT and GC rich regions, substitution errors and high half positive rate.

On the other hand, other SBS approaches such as 454 pyrosequencing and Ion Torrent (Thermo Fisher) use single-nucleotide Addition/Termination. 454 pyrosequencing could reach 400 bp while Ion Torrent can achieve 700 bp read lengths. However, although these technologies are faster and good for point of care, they also have many drawbacks including domination of insertion/deletion errors, and homopolymer region errors. They cannot be used to reveal long-range genomic or transcriptomic structure, and cannot do paired end sequencing.

Long-read sequencing approaches include two main types, synthetic long-read sequencing or real-time long-read sequencing. Synthetic pieced together long-read sequencing used by Illumina and 10X Genomics focuses on library preparation that leverages barcodes and allows computational assembly of large fragments. In fact, these technologies do not do actual long-reads, rather they do short-reads, in which the DNA pieces are organized using a barcoding approach, which helps eliminate some complexity during analysis, and which allows obtaining data similar to actual long-read methods. However, this approach has a very high cost due, in part, to its requiring even more coverage. The other type of long-read sequencing is real-time long-read sequencing, which has been used by Pacific Biosciences and Oxford Nanopore Technologies. Unlike synthetic long-read sequencing, real-time long-read sequencing does not rely on clonal population of amplified DNA and does not require chemical cycling. Nanopore’s technology has very high error rates around 30%, which also require very high coverage that contributes significantly to the cost. Using modified bases has also been particularly challenging for Nanopore’s technology, which has generated unique signals that makes the analysis even more complex. Pacific Biosciences can reach read lengths up to . However, due to high single-pass error rates around 15% for long reads, high coverage is required, which makes 1 Gb sequencing cost more than $1000 (see., e.g., Goodwin et al., Nat. Rev. Genet. 17:333-351; 2016). In addition, the thermal background present and excitation energy utilized by these methods damages the DNA polymerases used in the critical reactions, which ultimately limits the read lengths and applicability of this technology. In addition, as the luminescence generated is a generic spectrum independent of the nucleotide attached by the polymerase, pyrosequencing requires a cycle-based approach where each nucleotide is administered one by one collecting signal from all the binding events. This is followed with a washing cycle to remove the unbound nucleotides to administer the next nucleotide.

Since, a large majority of current technologies offer short read lengths (around 40-100 bases long) of nucleotides per unit, one of the most challenging problem lies in alignment of small pieces of sequences into one large meaningful sequence, and analyzing high coverage data and the post-processing of the loads of generated data with complicated algorithms using powerful super computers. Newer generation single molecule based sequencing technologies can potentially address this issue. However, each of these prior art technologies have high error rates requiring high coverages (multiple reads of the same region of a sequence) often around 30X to 100X in order to obtain a reliable data.

Accordingly, there is a need for improved methods for nucleic acid sequencing.

### SUMMARY

Provided herein are methods for sequencing a nucleic acid template comprising:
providing a sequencing mixture comprising (i) a polymerase enzyme, (ii) a luminescence enzyme, (iii) a template nucleic acid and primer, and (iv) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group (e.g., PPi-LS) that is cleavable by the polymerase, and each type of nucleotide-conjugate-analog has a different luminescent-substrate attached thereto, wherein the luminescent-substrate-attached-leaving-group is cleaved upon polymerase-dependent binding of a respective nucleotide-conjugate-analog to the template strand;
carrying out nucleic acid synthesis such that a plurality of nucleotide-conjugate-analogs are added sequentially to the template whereby: a) a nucleotide-conjugate-analog associates with the polymerase, b) the nucleotide-conjugate-analog is incorporated on the template strand by the polymerase when the luminescent-substrate-attached-leaving-group on that nucleotide-conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce nucleotide-specific-luminescence for a limited period of time; and
detecting nucleotide-specific-luminescence signal (light) while nucleic acid synthesis is occurring, and using nucleotide-specific-luminescence signal detected during each discreet luminescence period to determine a sequence of the template nucleic acid.

Accordingly, provided herein is a method for real-time or cycle based single molecule sequencing, LASH (Luminescence Activation By Serial Hybridization). In this approach, there is a luminescent-substrate attached to a phosphate, e.g., the gamma phosphate, and the like, of the various nucleotides (e.g., dNTPs). Each nucleotide has a luminescent-substrate with different spectra. Polymerase accepts this modified nucleotide as a substrate. Each time polymerase binds complementary nucleotide to the template strand, it releases pyrophosphate with the luminescent-substrate attached and unique to the nucleotide that was incorporated in to the template strand by polymerase.

The pyrophosphate modified with luminescent-substrate attached (referred to herein as luminescent-substrate-attached-leaving-group or PPi-LS) has unique spectrum for each different nucleotide, and interacts with a luminescence enzyme (i.e. firefly luciferase, click beetle luciferase, gaussian luciferase, renilla luciferase, microperoxidase, myeloperoxidase, horseradish peroxidase, catalase, xanthine oxidase, bacterial peroxidase from Arthromyces ramosus, alkaline phosphatase, β-D-galactosidase and b-glucosidase in the presence of indoxyl conjugates as substrates, lactate oxidase, acylCoA synthetase and acylCoA oxidase, diamine oxidase, 3-a hydroxysteroid deshydrogenase or glucose-6-phosphate deshydrogenase, and the like) to produce a short-lived nucleotide-specific-luminescent signal corresponding to the base or nucleotide incorporated in to the template strand. Real-time sequencing is achieved by reading the short-lived pulses having unique spectra, which correspond to the respective nucleotides that were attached.

A key advantage of the invention sequencing methods (also referred to herein as the LASH sequencing method; Luminescence Activation by Serial Hybridization) is that the polymerase enzyme is not damaged in the invention reaction conditions, such as by being attached to a particular surface, or being subj ect to multiple exposures of external light excitation used to generate signal; as occurs with existing methods. The invention methods do not require a major modification to the polymerase, or its attachment to a surface as well as its exposure to external light sources that pressure polymerase from performing its native chain elongation function. This advantageously results in a longer functioning polymerase able to reach very long read lengths with as much accuracy high fidelity as occurs in its native environment; with much less coverage required than existing methods.

For example, in particular embodiments of the present invention, either a single polymerase or a plurality of polymerases are confined with the sequencing reaction mixture, such as for example in a single droplet, or the like, wherein the polymerase(s) is not subject to external light excitation to generate the dNTP incorporation signal to be detected.

The invention methods have a variety of uses including whole genome sequencing, SNP-variant detection, and the like. One advantage of the invention methods over existing methods is the utilization of modified nucleotide-conjugate-analogs having luminescent-substrates attached thereto (e.g., luminescent-substrate-attached-nucleotides) in a nucleotide-specific-luminescence reaction (for example using a marine luciferase and coelenterazine, or bacterial luciferase and FMNH2, and the like) to generate a controlled, uniquely defined, discreet and/or transient limited nucleotide-specific-luminescence signal. It has surprisingly been found that the luminescent-substrate-attached-leaving-group can function in a nucleotide-specific-luminescence reaction using a marine luciferase and coelenterazine, or bacterial luciferase and FMNH2, and the like. Another advantage of the invention methods over existing methods is the reduction in light intensity utilized by the luminescence reaction, such that damage to the DNA polymerase does not occur as most conventional methods require external excitation with high intensity light that denatures polymerases eventually. For example, the luminescence light intensity generated can be reduced compared to existing sequencing methods by at least 5-fold, 10-fold, 25-fold, 50-fold, 75-fold, 100-fold up to at least 1,000-fold. In particular embodiments, the reduction in light intensity can be at least 5-fold, 10-fold, 25-fold, 50-fold, 75-fold, 100-fold, 200-fold, 300-fold, 400-fold, 500-fold, 600-fold, 700-fold, 800-fold, 900-fold, 1000-fold, 2000-fold, and the like. This advantage results in the longer functioning of the DNA polymerase, thereby producing longer read lengths.

In particular embodiments, the invention method provided herein is a single molecule sequencing technology based on monitoring the results of individual polymerase enzymes as they incorporate dNTPs sequentially. In a particular embodiment, the invention encompasses a process where each time polymerase incorporates a dNTP, or analog thereof, complementary to the template, a nucleotide-specific-luminescence signal is transiently, uniquely and/or discreetly generated during the incorporation process, wherein such nucleotide-specific-luminescence signal is caused by a transient, unique and/or discreet luminescence reaction. In other words, the luminescence reaction causes the respective luminescence-substrate, via the excitation spectra and the like, to emit a detectable signal for a limited amount of time specific for, and corresponding to, that particular dNTP. The process repeats for the next dNTP incorporation (FIG. 1).

More particularly, each time a polymerase incorporates a modified deoxyribonuleoside triphosphate (dNTP) nucleotide-conjugate-analog to the strand complementary to the template DNA, a luminescence signal specific to the type of the nucleotide attached is generated (e.g., a nucleotide-specific-luminescence signal). There are five types of dNTPs, namely deoxyadenosine triphosphate (dATP), deoxyguanosine triphosphate (dGTP), deoxycytidine triphosphate (dCTP), deoxythymidine triphosphate (dTTP), and deoxyuradine triphosphate (dUTP). Four or five of these dNTPs are used in the template directed nucleic acid synthesis reaction to identify (i.e., call) its complement (e.g., adenine, guanine, cytosine, or thymine) in the template nucleic acid strand, thereby sequencing the template nucleic acid strand.

Each modified nucleotide-conjugate-analog generates a unique luminescence signal (e.g., wavelengths of 411, 417, 428, 440, 484, 509 nm, and the like) from the attached luminescence substrate while they are being attached to the complementary strand by the polymerase enzyme. Either dTTP or dUTP or any combination of both can be used in a nucleic acid synthesis chain elongation reaction to call (i.e., identify) the complementary adenine (ATP) in the sequence. If both modified dTTP and dUTP analogs are used in the reaction, they can each have the same luminescence substrate attached thereto producing the same wavelength signal; or each can have a discreet luminescence substrate attached thereto. Upon the completion of attachment of the nucleotide-conjugate-analog to the 3′ moiety of the previously attached nucleotide-conjugate-analog, the luminescence generated by the luminescence-substrate-attached-leaving-group is detected by an appropriate luminescence sensor and/or detection device and then, in some embodiments, it is subsequently rapidly terminated by decay of luminescence reaction for that respective dNTP incorporation. In other words, each dNTP incorporated into the template strand results in a discreet, limited-period pulse of light (luminescence signal) that is unique and indicative of that respective dNTP incorporation event, which permits the calling or identification of the particular complementary base in the template nucleic acid being sequenced.

In other embodiments, the luminescence generated by the luminescent-substrate-attached-leaving-group is amplified and detected by an appropriate luminescence sensor and/or detection device and then, in some embodiments, it is subsequently rapidly terminated by decay of luminescence reaction for that respective dNTP incorporation.

Sequencing of the desired template nucleic acid is achieved by detecting the luminescence generated each time a nucleotide is added to the complementary strand revealing the type of nucleotide. Therefore, each specific nucleotide attachment generates a short peak of a luminescence signal that can be detected by a luminescence sensor. As a result, a data array of succeeding, sequential wavelength signals is produced, which can be converted into a corresponding data array of nucleotide sequence.

An advantage provided by the invention methods disclosed herein lies in its simplicity and innovative chemistry that significantly reduces background signal during detection thereby improving sensitivity. In accordance with the present invention methods, less modification of the reaction conditions involving reagents and enzymes improves specificity, efficiency and rate. Also, in accordance with the present invention methods, polymerase operates in near ideal conditions, and is contemplated to reach very long read lengths around tens of thousands of bases per DNA polymerase molecule by utilizing high sensitivity and specificity together with requiring significantly less post-processing and analysis of the data produced. The combined features of the invention methods disclosed herein reduces the cost both for the respective devices and each run, while achieving high specificity in addition to decreasing the time per test considerably compared to competing technologies. Accordingly, the disclosed invention methods and systems allow realization of very low cost and real-time nucleic acid sequencing systems without adversely affecting specificity.

Also provided herein are methods for detecting the presence of a target nucleic acid sequence in a sample comprising:
providing an elongation mixture comprising (i) a polymerase enzyme, (ii) a luminescence enzyme, (iii) a template nucleic acid sample, (iv) a primer-probe that hybridizes to (e.g., that is complementary to) a particular target nucleic acid sequence, and (v) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has the same, or different, luminescent-substrate attached thereto, wherein the luminescent-substrate-attached-leaving-group is cleaved upon polymerase-dependent binding of a respective nucleotide-conjugate-analog to the template strand;
carrying out nucleic acid elongation synthesis such that a plurality of nucleotide-conjugate-analogs are added sequentially to the template if the primer-probe hybridizes to the target nucleic acid sequence, whereby: a) a nucleotide-conjugate-analog associates with the polymerase, b) the nucleotide-conjugate-analog is incorporated on the template strand by the polymerase when the luminescent-substrate-attached-leaving-group on that nucleotide-
conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce luminescence; and
detecting light from the luminescence while nucleic acid synthesis is occurring, whereby detection of light indicates the presence of the particular target nucleic acid sequence.

In particular embodiments, the amount of target nucleic acid is quantified. In one embodiment, the amount of target nucleic acid is quantified based on the intensity of the luminescence. In a particular embodiment, each type of nucleotide-conjugate-analog has the same luminescent-substrate attached thereto. In particular embodiments, a plurality of polymerase enzymes are used.

An advantage, of the invention target nucleic acid sequence detection and/or quantification methods, is detection of a particular sequence without the need for temperature cycling, or substantial increase of the copy number of DNA. Using the invention methods, in certain embodiments, the light produced from the hybridization of the primer-probe to its target nucleic is essentially continuous based on the length of the target nucleic acid template, resulting in a chain-elongation-light-emitting reaction instead of an exponential increase of the copy number.

Another advantage of the invention light-signal target nucleic acid detection methods provide herein, is that they are much quicker than PCR in providing a detectable, actionable signal. For example, a typical PCR typically has up to around 30-40 thermal-cycles, where each cycle takes several minutes to complete leading to a total run duration of at least one to a few hours. One can do shorter runs with PCR, but give up specificity; and those shorter run cases are very limited in terms of primer, probe and template configurations. In contrast, the invention light-signal detection methods for detecting and/or quantifying target nucleic acid sequences (e.g. LACES) starts to produce a detectable signal as soon as elongation begins. In some embodiments, the initial signal that is produced very early (e.g., in a matter of minutes, and the like) is the highest and the most specific signal relative to the later signal. Therefore, the evolution of the signal produced by LACES can be described by a rapid initial rise followed by a long decay; whereas with quantitative PCR, it is an exponential increase that becomes detectable after many cycles and a much longer time-frame, eventually reaching a plateau. More particularly, LACES provides a very specific signal in the initial rapid rise period that occurs much earlier compared to qPCR without giving up specificity.

For example, in particular embodiments of the present invention, either a single polymerase or a plurality of polymerases are confined with the nucleic acid chain elongation reaction mixture (e.g, either in a bulk reaction or in a single droplet), wherein the polymerase(s) is not subject to external light excitation to generate the dNTP incorporation signal to be detected.

Also provided herein are luminescent-substrate-nucleotide-conjugate-analogs, comprising a deoxyribonucleotide (dNTP), or analog thereof; and a luminescent-substrate attached thereto. In certain embodiments, the nucleotide (dNTP) within the luminescent-substrate-nucleotide-conjugate-analogs are modified nucleotide analogs. In particular embodiments, the dNTP is selected from the group consisting of: dATP, dTTP, dGTP, dCTP and dUTP, dATPαS, dGTPαS, dCTPαS, dTTPαS and dUTPαS. In certain embodiments, the nucleotide-conjugate-analog is capable of being a substrate for the polymerase and for the selective cleaving activity.

In one embodiment, the nucleotide-conjugate-analog is a nucleoside polyphosphate having three or more phosphates in its polyphosphate chain with a luminescent substrate attached to the portion of the polyphosphate chain that is cleaved upon incorporation into a growing template directed strand. In particular embodiments, the polyphosphate is a pure polyphosphate (—O—PO3—), a pyrophosphate (PPi), or polyphosphate having substitutions therein. In further embodiments, the luminescent-substrate is selected from coelenterazine, FMNH2, or analogs thereof. In a particular embodiment, the luminescent-substrate is attached to a terminal phosphate. In other embodiments, when the PPi luminescent-substrate-attached-leaving-group is generated by the polymerase when the luminescent-substrate nucleotide-conjugate is incorporated into the template strand, the luminescent-substrate-attached-pyrophosphate or luminescent-substrate-attached-leaving-group is able to be combined with the respective luciferase.

In a particular embodiment, the PPi luminescent-substrate-attached-leaving-group is selected from PPi-LS, PPi-C; or PPi-FMNH2. In further embodiments, the nucleotide-conjugate-analog has a unique luminescent signal. In a particular embodiment, the luminescence signal is a wavelength selected from the range 250 nm - 750 nm. In another embodiment, the luminescence signal is a wavelength selected from the group consisting of: 411, 417, 428, 440, 484, and 509 nm.

Also provided herein is a chain-elongation set of nucleotide-conjugate-analogs comprising at least 4 distinct a deoxyribonucleotides (dNTPs), such that the chain-elongation set can be incorporated into template directed synthesis of a growing nucleic acid strand. In one embodiment, each respective dNTP, or analog thereof, is modified using a different, unique luminescent substrate relative to the other dNTPs, such that each time a polymerase incorporates a modified deoxyribonuleoside triphosphate (dNTP) nucleotide-conjugate-analog to the strand complementary to the template DNA, a luminescent signal specific to the respective nucleotide attached is generated. In another embodiment, if both modified dTTP and dUTP analogs are used in the reaction, they can each have the same luminescent substrate attached thereto producing the same wavelength signal; or each can have a discreet luminescent substrate attached thereto.

In particular embodiments, the dNTP is selected from the group consisting of: dATP, dTTP, dGTP, dCTP and dUTP, dATPαS, dGTPαS, dCTPαS, dTTPαS and dUTPαS. In further embodiments, the luminescent-substrate is selected from coelenterazine, FMNH2, or analogs thereof. In yet further embodiments, the chain-elongation set of nucleotide-conjugate-analogs can be selected from Coelenterazine-dNTP Conjugate 1 (FIG. 7); Coelentarazine-dNTP Conjugate 2 (FIG. 8); or Coelentarazine-dNTP Conjugate 3 (FIG. 9).

### BRIEF DESCRIPTION OF THE DRAWINGS

FIG. 1A shows a general illustration of one exemplary embodiment of the invention sequencing method using four different luminescent-substrate analogs for each nucleotide catalyzed by the same luminescence enzyme.

FIG. 1B shows a general illustration of one exemplary embodiment of the invention sequencing method using four different luminescent-substrate-enzyme systems for each nucleotide, such that there are four different luminescent-substrate analogs for each nucleotide catalyzed by four different, respective luminescence enzymes. Also contemplated are additional embodiments using only 2 or 3 different luminescent-substrate-enzymes for the 4 different luminescent-substrate analogs on the 4 modified nucleotides (e.g., A, T, G and C).

FIG. 2A shows a general illustration of one exemplary embodiment of the invention sequencing method using coelenterazine analogs and either or both of Renilla Luciferase or Gaussia Luciferase: DNA Polymerase uses dNTPs modified with the respective coelenterazine luminescence substrate as building blocks for the template strand (e.g., dNTP-C1). Upon binding to polymerase, the pyrophosphate containing a coelenterazine luminescent-substrate (e.g., luminescent-substrate-attached-leaving-group or PPi-C1) is cleaved off for later reactions.

FIG. 2B shows the polymerase-dependent binding of a respective nucleotide-conjugate-analog, having a coelenterazine analog luminescence-substrate attached therein, to the template strand and the cleaving of the pyrophosphate-Cl leaving group (e.g., luminescence-substrate-attached-leaving-group) that has the coelenterazine analog attached (PPi-Cl), which will next interact with a luciferase (e.g, Renilla Luciferase, Gaussia Luciferase, or the like).

FIG. 2C shows the reagents, luminescence-substrate-attached-leaving-group (PPi-Cl), and Renilla and/or Gaussia luciferase, for the luminescence reaction set forth herein. The interaction of these reagents in the luminescence reaction is shown, from which the coelenterazine-attached-pyrophosphate (PPi-Cl) will luminesce. There is a unique luminescence substrate (e.g., coelenterazine or a flavin analog) for each type of nucleotide-conjugate-analog dNTP, such that each type of nucleotide produces a unique luminescing signal corresponding that respective base.

FIG. 3A shows a general illustration of one exemplary embodiment of the invention sequencing method using flavin mononucleotide analogs (FMNH2 analogs) and a Bacterial Luciferase: DNA Polymerase uses dNTPs modified with the respective coelenterazine luminescence substrate as building blocks for the template strand (e.g., dNTP-FMNH2). Upon binding to polymerase, the pyrophosphate containing a coelenterazine luminescent-substrate (e.g., luminescent-substrate-attached-leaving-group or PPi-FMNH2) is cleaved off for later reactions.

FIG. 3B shows the polymerase-dependent binding of a respective nucleotide-conjugate-analog, having a flavin mononucleotide analog (FMNH2 analog) luminescence substrate attached therein, to the template strand and the cleaving of the pyrophosphate-FMNH2 leaving group (e.g., luminescence-substrate-attached-leaving-group) that has the FMNH2 analog attached (PPi-FMNH2), which will next interact with a bacterial luciferase, or the like.

FIG. 3C shows the reagents, luminescence-substrate-attached-leaving-group (PPi-FMNH2), and bacterial luciferase, for the luminescence reaction set forth herein. The interaction of these reagents in the luminescence reaction is shown, from which the FMNH2-attached-pyrophosphate (PPi-FMNH2) will luminesce. There is a unique luminescence substrate (e.g., coelenterazine or a flavin analog) for each type of nucleotide-conjugate-analog dNTP, such that each type of nucleotide produces a uniquely detectable luminescing signal corresponding that respective base.

FIG. 4 shows an exemplary strategy for the large scale synthesis of coelenterazine.

FIG. 5 shows the synthesis of coelenterazine analog-1.

FIG. 6 shows the synthesis of coelenterazine analog-2.

FIG. 7 shows the synthesis of coelenterazine-dNTP conjugate-1.

FIG. 8 shows the synthesis of coelenterazine-dNTP conjugate-2.

FIG. 9 shows the synthesis of coelenterazine-dNTP conjugates 1, 2 and 3.

FIG. 10A shows an embodiment of confining the LASH reaction reagents in a confinement area corresponding to a droplet; and shows a single target nucleic acid template in a sequence mixture having a plurality of polymerases and a plurality of primers.

FIG. 10B shows an embodiment of confining the LASH reaction reagents in a confinement area corresponding to a droplet; and shows a sequence mixture having plurality of target nucleic acid templates, a plurality of polymerases and a single primer, such that only a single target nucleic acid template is sequenced.

FIG. 10C shows an embodiment of confining the LASH reaction reagents in a confinement area corresponding to a droplet; and shows a single self-priming target nucleic acid template in a sequence mixture having a plurality of polymerases.

FIG. 11A shows the configuration where the primer is attached to a solid surface substrate, for subsequent binding of the target template nucleic acid.

FIG. 11B shows the configuration where the target nucleic acid template is attached to a solid surface substrate, for subsequent binding of the primer.

FIG. 12A shows the embodiment of initiating the invention sequencing methods using a plurality of polymerases on a single target nucleic acid template.

FIG. 12B shows an embodiment where the sequencing of the target template is substantially continuous because as the polymerase that starts synthesizing the complementary strand traverses its typical read length, then falls off or dissociates from template, another of the many other polymerases in the reaction mixture immediately binds to the template and continues the complementary strand sequencing synthesis.

FIG. 13 shows an embodiment where numerous identical primers are bound to a substrate each at discreet loci, which can be in either a single overall reaction chamber, or in individual discreet reaction chambers. These primers bind essentially the same target template nucleic acid.

FIG. 14 shows an embodiment where numerous different (mutually exclusive) primers are bound to a substrate each at discreet loci, which can be in either a single overall reaction chamber, or in individual discreet reaction chambers. These primers bind different, mutually exclusive target template nucleic acids.

FIG. 15 shows a simplified schematic of the biochemical process of dNTP incorporation into a template strand.

FIG. 16A shows a general illustration of one exemplary embodiment of the invention sequencing method using flavin mononucleotide analogs (FMNH2 analogs) and a Bacterial Luciferase.

FIG. 16B shows the polymerase-dependent binding of a respective nucleotide-conjugate-analog, having a flavin mononucleotide analog (FMNH2 analog) luminescence substrate attached therein, to the template strand and the cleaving of the pyrophosphate-FMNH2 leaving group (e.g., luminescence-substrate-attached-leaving-group) that has the FMNH2 analog attached (PPi-FMNH2), which will next interact with a bacterial luciferase, or the like.

FIG. 16C shows the beginning of the oxidoreductase/Luciferase signal amplification loop where the luminescence-substrate-attached-leaving-group (PPi-FMNH2) is oxidized (depicted by FMN*) by bacterial luciferase in the luminescence signalling reaction set forth herein.

FIG. 16D shows the oxidoreductase reaction where the oxidized luminescence substrate FMN* is reduced back to FMNH2 on the pyrophosphate leaving group to loop back into the luminescence reaction of FIG. 16C, thereby completing the oxidoreductase/Luciferase enzymatic loop.

### DETAILED DESCRIPTION

Provided herein are methods for sequencing a nucleic acid template, wherein said methods comprise:
providing a sequencing mixture comprising (i) a polymerase enzyme, (ii) a luminescence enzyme (iii) a template nucleic acid and primer, and (iv) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has a different luminescent-substrate attached thereto, wherein the luminescent-substrate-attached-leaving-group is cleaved upon polymerase-dependent binding of a respective nucleotide-conjugate-analog to the template strand;
carrying out nucleic acid synthesis such that a plurality of nucleotide-conjugate-analogs are added sequentially to the template whereby: a) a nucleotide-conjugate-analog associates with the polymerase, b) the nucleotide-conjugate-analog is incorporated on the template strand by the polymerase when the luminescent-substrate-attached-leaving-group on that nucleotide-conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce nucleotide-specific-luminescence for a limited period of time; and
detecting nucleotide-specific-luminescence signal (light) while nucleic acid synthesis is occurring, and using nucleotide-specific-luminescence signal detected during each discreet luminescence period to determine a sequence of the template nucleic acid.

As used herein, the phrase “luminescence enzyme,” or grammatical variations thereof, e.g., “luminescent enzyme,” and the like, refers to any molecule or enzyme that can catalyze a luminescence substrate (or luminescent substrate) within a luminescence-substrate-attached-leaving-group (i.e., PPi-LS) in a luminescence reaction. Both luminescence-substrate and luminescent-substrate are use herein interchangeably; as well as luminescence enzyme and luminescent enzyme. Exemplary luminescence enzymes for use herein include luciferases, such as for example, marine or bacterial luciferases, and the like. In other embodiments, exemplary luminescence enzymes include photoproteins, such as aequorin, obelin, and the like. For example, in one embodiment when coelenterazine is used as the luminescent-substrate, a marine luciferase, such as for example, Renilla Luciferase, Gaussia Luciferase, and the like; or any combination thereof is used in the luciferase reaction. In other embodiments using coelenterazine, a photoprotein such as for example, aequorin, obelin, and the like; or any combination thereof is used in the reaction mixture. Also contemplated herein, is the use of any combination of luciferases and photoproteins in the luciferase reactions, so long as the overall luminescence reactions are able to distinguish the respective luminescence signal (e.g., spectra) from each of the uniquely modified nucleotide-conjugate-analogs.

In other embodiments when FMNH2 is used as the luminescent-substrate, suitable luminescence enzymes are bacterial luciferases obtained generally from a variety of bacterial genera, including Vibrio and Photobacterium. More particularly, bioluminescence luciferase species suitable for use herein include those obtained from, for example, Vibrio harveyi, Vibrio fischeri (commercially available from Millipore, SIGMA), Photobacterium fischeri, Photobacterium phosphoreum, P. leiognathi, P. luminescens and the like.

As used herein, the phrase “luminescence substrate,” “luminescent substrate,” or grammatical variations thereof, refers to any a molecule or moiety that can be attached to any location on a nucleotide, such that upon incorporation of that modified nucleotide into an elongating nucleic acid strand, a luminescence signal is generated in the presence of a luminescence enzyme as a result of a luminescence reaction. Suitable luminescence substrates for use herein, include coelenterazine and analogs thereof, flavin mononucleotide (FMNH₂) or analogs thereof, luminol, isoluminol and their derivatives, acridinium derivatives, dioxetanes, peroxyozalic derivatives, and the like.

Coelenterazine is a substrate involved in bioluminescence catalyzed by variety of marine luciferases including Renilla reniformis luciferase (Rluc), Gaussia luciferase (Gluc), and photoproteins, including aequorin, and obelin. One important advantage provided by coelenterazine is that it does not require ATP as a cofactor in its luciferase reaction, which is different from the co-factor requirements of other luciferases like firefly and click beetle luciferases. Another advantage provided by Coelenterazine, is that its bioluminescence light spectrum can be adjusted by chemical modification. Accordingly, suitable coelenterazine analogs for use herein as the luminescence substrates are commercially available from a variety of sources, including Molecular Probes (Eugen, OR, Biotium (Freemont, CA), and the like. For example, coelenterazine analogs available from Molecular Probes (Eugene, OR), including C-2944 (native); C-14260 (coelenterazine cp); C-6779 (coelenterazine f); C-6780 (coelenterazine h); C-14261 (coelenterazine hcp); C-6776 (coelenterazine n). The coelenterazine analogs available from Biotium include Catalog Nos: No. 10110 (native Coelenterazine); No. 10124 (Coelenterazine 400a); No. 10112 (Coelenterazine cp); No. 10114 (Coelenterazine f); No. 10117 (Coelenterazine fcp); No. 10111 (Coelenterazine h); No. 10113 (Coelenterazine hcp); No. 10121 (Coelenterazine i); 10116 (Coelenterazine ip); No. 10122 (Methyl Coelenterazine, 2-methyl analog); No. 10115 (Coelenterazine n); and the like. See Table 1 for the luminescent properties of these Coelenterazine analogs with Renilla Luciferase.

TABLE 1

Luminescent Properties of Coelenterazine Analogs with Renilla Luciferase*

| Analog | λₑₘ (nm) | Total Light (%) | Initial Intensity (%) |
|---|---|---|---|
| Native | 475 | 100 | 45 |
| 400a | 400 | | |
| Cp | 470 | 23 | 135 |
| E | 418, 475 | 137 | 900 |
| F | 473 | 28 | 45 |
| H | 475 | 41 | 135 |
| N | 475 | 47 | 900 |

*Data from Biochem. Biophys. Res. Commun. 233, 349 (1997)

See Table 2 for the luminescent properties of these Coelenterazine analogs with the photoprotein Aequorin.

TABLE 2

Luminescent Properties of Coelenterazine Analogs with Apoaequorin*

| Analog | λₑₘ (nm) | Relative luminescence capacity | Relative intensity | Half-rise time(s) |
|---|---|---|---|---|
| native | 465 | 1.0 | 1.00 | 0.4-0.8 |
| cp | 442 | 0.95 | 15 | 0.15-0.3 |
| e | 405, 465 | 0.50 | 4 | 0.15-0.3 |
| f | 473 | 0.80 | 18 | 0.4-0.8 |
| fcp | 452 | 0.57 | 135 | 0.4-0.8 |
| h | 475 | 0.82 | 10 | 0.4-0.8 |
| hcp | 444 | 0.67 | 190 | 0.15-0.03 |
| i | 476 | 0.70 | 0.03 | 8 |
| ip | 441 | 0.54 | 47 | 1 |
| n | 467 | 0.26 | 0.01 | 5 |

*Data from Biochem. J. 261, 913 (1989)

Other suitable coelenterazine analogs for use herein are set forth as compounds 1-120 in Jiang et al., Photochem. Photobiol. Sci. 2016, 15, 4660480; set forth as DeepBlueC, and compounds B1-B12 in Jiang et al., Org. Biomol. Chem. 2017, 15, 7008-7018; and compounds CoelPhos, 2-Bno-TEG-CTZ, and 6-BnO-TEG-CTZ in Lindberg et al., Chem. Sci., 2013, 4, 4395-4400; each of which are incorporated by reference in their entirety for all purposes.

Bacterial luciferase catalyzes the oxidation of FMNH₂ utilizing oxygen (O₂) and reduced fatty acid (RCHO) and releases an analog of oxidized form of flavin mononucleotide (FMN) and fatty acid (RCOOH) using the well-known mechanism set forth in Mitchell et al., J. Biol. Chem., Vol. 244, No. 10, 2572-2576 (1969). Molecular oxygen is consumed in the reaction, reminiscent of part of an electron transport system in aerobic respiration, except that instead of serving as the final electron acceptor, oxygen interacts with the enzyme luciferase and FMNH2 to generate light. Short-lived luminescence is generated as a result of this process each time a new nucleotide is attached to the nucleic acid template strand. It has been found that FMN accommodates various functionalizations that result in spectral shifts in the luminescence. See, for example, the flavin mononucleotide analogs set forth in Mitchell et al., J. Biol. Chem., Vol. 244, No. 10, 2572-2576 (1969); Salzmann et al., J. Phys. Chem. A 2009, 113, 9365-9375; Eckstein et al., Biochemistry, 1993, 32, 404-4111; and the like; each of which journal references are incorporated by reference herein in their entirety for all purposes. Exemplary flavin mononucleotide analogs known in the art for use herein, include: 1-deazariboflavin; 5-deazariboflavin; 7,8-didemethyl-isopropylriboflavin; 8-isopropylriboflavin; the 8-substituted 3,7,10-trimethylisoallox-azines, 3-methyl-lumiflavin, 3,7,10-trimethylisoalloxazine, and 3,7-dimethyl-8-methoxy-10-ethylisoalloxazine; 3-Methyl-4a,5-propano-4a,5-dihydroisoalloxazine; 3.7.10- Trimethyl-4a,5-propano-4a,5-dihydroisoal!ox-azine; 3.7.10-Trimethyl-8-chloro-4a,5-propano-4a,5-dihydro-isoalloxazine; 3.7.10-Trimethyl-8-methoxy-4a,5-propano-4a,5-di-hydroisoalloxazine and 3,7,10-Trimethyl-8-amino-4a,5 ·propano-4a,5-dihydroisoalloxazine; FAD; Riboflavin; Iso-FMN; 2-Thio-FMN; 2-Morpholino, 2-desoxy FMN; 2-(Beta-Hydroxyethyl amino)-FMN; 3-Acetyl-FMN; 2-Phenylimino-FMN; Isoriboflavin; Tetraacetyllisoriboflavin; Lumiflavin-3-acetic acid; Neutral red; and the like.

In one embodiment, a different analog of FMNH2 is attached to each of the four or five nucleotides (e.g., dNTPs), such that each analog of FMNH2 has a different nucleotide-specific-luminescence spectra (e.g., wavelength signal) in the luminescence reactions, corresponding specifically the type of the nucleotide that is attached. In other words, each nucleotide can be modified with a different FMN analog leading to different luminescence spectra specific to the nucleotide upon interaction with bacterial luciferase. FMNH2 has a phosphate group at one end this group can be attached as a terminal group to the phosphate chain of a particular nucleotide. Those of skill in the art will recognize that this can be done either chemically or enzymatically using an enzyme such as ATP synthase, or the like.

As used herein, the phrase “sequencing mixture” refers to the components that are used to carry out the invention single molecule sequencing reactions. In one embodiment, the sequencing mixture includes (i) a polymerase enzyme, (ii) a luminescence enzyme, (iii) a template nucleic acid and primer, and (iv) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has a different luminescent-substrate attached thereto.

In accordance with the present invention, the sequencing mixture used provides the following advantages in the invention sequencing methods over previous sequencing methods: the polymerase employed functions in its ideal state; there is no need to modify a polymerase enzyme; the use of high nucleotide (e.g., dNTP) concentrations results in optimum efficiency; generates only very-low intensity, discreet and limited period of detectable light signal via the luminescence reaction, which advantageously reduces the denaturing of the polymerase enzyme; provides essentially no (or very low) background, which improves specificity and sensitivity of the base calling; does not require sophisticated optics or nanostructured chip design, which reduces cost. The invention methods also provide high specificity, which reduces the need for high coverage. As a short-lived signal is generated per event successively, this approach does not rely on only one polymerase molecule. Thus, if the polymerase falls from the template oligonucleotide after several successive base attachments (e.g, 10, 100, 1,000 or 1,000,000 succesive base attachments), a new polymerase binds to wherever the prior polymerase fell off, to keep attaching bases continuously. This way, the read-length is virtually unlimited. With this approach, read lengths as long as the entire gene length (several 10 Kbs) or spanning several gene lengths (several 100 Kbs) or even large segments such as several Mbs is possible. This not only makes new applications possible but also dramatically reduces computer processing required relative to prior art methods.

As used herein, the phrase “polymerase-luminescence reagent solution,” or grammatical variations thereof, or “reagent solution” refers to the mixture of components necessary for carrying out the template directed synthesis of a growing nucleic acid, and the luminescence reaction. In one embodiment, the dNTPs are modified with coelenterazine and/or coelenterazine analogs as the luminescent-substrate. In this embodiment, the polymerase-luminescence reagent solution for use with a polymerase, e.g., DNA pol I, and the luminescence-enzyme, includes a marine luciferase (e.g., Renilla reniformis luciferase (Rluc), Gaussia luciferase (Gluc), and the like) and suitable concentrations of modified dNTP analogs, e.g., coelenterazine-modified nucleotide-conjugate-analogs described herein. In some embodiments, the nucleotide-conjugate-analogs can have 4 or more phosphates therein and the coelenterazine analog is attached to the terminal phosphate. For example, nucleotide-conjugate-analogs having 4, 5, 6, 7, 8, 9, 10, 11, 12, 13, 14, 15, 16, 17, 18, 19, 20, or more phosphates are contemplated herein, with the coelenterazine analog attached to the terminal phosphate.

In another embodiment, the dNTPs are modified with an analog of reduced form of flavin mononucleotide (FMNH2) as the luminescent-substrate. In particular embodiments, the flavin mononucleotide or analog thereof is attached to the terminal phosphate of the deoxynucleotide. In this embodiment, the polymerase-luminescence reagent solution for use with a polymerase, e.g., DNA pol I, and the luminescence-enzyme, includes a bacterial luciferase and suitable concentrations of modified dNTP analogs, e.g., FMNH2-modified nucleotide-conjugate-analogs described herein. As set forth above, in some embodiments, the nucleotide-conjugate-analogs can have 4 or more phosphates and the FMNH2 analog is attached to the terminal phosphate. For example, nucleotide-conjugate-analogs having 4, 5, 6, 7, 8, 9, 10, 11, 12, 13, 14, 15, 16, 17, 18, 19, 20, or more phosphates are contemplated herein, with the FMNH2 analog attached to the terminal phosphate.

In another embodiment contemplated herein, the luminescence substrate can be attached to any other location on the respective dNTP, so long as that upon incorporation of that modified dNTP analog into the elongating sequence, the luminescence substrate is able to combine with the luminescence enzyme to undergo a nucleotide-specific-luminescence reaction, generating the nucleotide-specific-luminescence signal. In other embodiments, other locations on the dNTPs suitable for attaching the luminescence substrate include the base and sugar.

As used herein the phrase “luminescence reaction” refers to any reaction that can produce the emission of light that does not derive all, or solely derive, energy from the temperature of the emitting body (i.e., emission of light other than incandescent light). Luminescence can be caused by chemical reactions, electrical energy, subatomic motions or stress on a crystal. “Luminescence” includes, but is not limited to, fluorescence, phosphorescence, thermoluminescence, chemiluminescence, electroluminescence and bioluminescence. “Luminescent” refers to an object that exhibits luminescence. In particular embodiments, the light is in the visible spectrum. However, the present invention is not limited to visible light, but includes electromagnetic radiation of any frequency. In particular embodiments, the luminescence reaction employed herein is caused by the luminescence enzyme, luciferase (e.g., a marine or bacterial luciferase) catalyzing the luminescence-substrate, e.g, coelenterazine or analogs thereof, or flavin mononucleotide (FMNH2) or analogs thereof, to produce luminescence.

For example, in one embodiment, the iterative sequencing cycle contemplated herein involves a first dNTP incorporation reaction, which results in the production of a luminescence-substate-attached-leaving-group (LSALG or PPi+LS). In a second reaction, the luminescence reaction, luciferase catalyzes LSALG to generate light. Thus, after each respective dNTP analog is incorporated, a quantum of light is generated for each molecule of luminescence-substrate-attached pyrophosphate (PPi + C or PPi + FMNH2) in solution. The invention is not limited to the type of luciferase used. Although certain disclosed embodiments utilize marine or bacterial luciferases, any luciferase known in the art that can catalyze a luminescence-substrate described herein may be used in the disclosed methods.

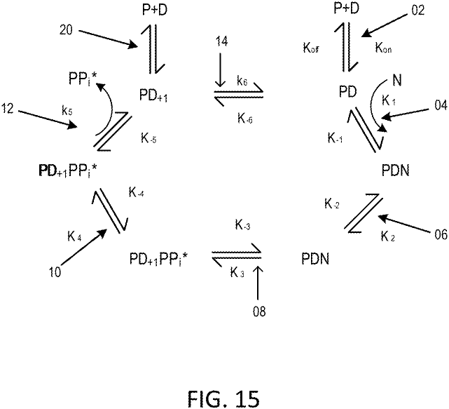

As used herein a “polymerase enzyme” refers to the well-known protein responsible for carrying out nucleic acid synthesis. A preferred polymerase enzyme for use herein is a DNA polymerase. In natural polymerase mediated nucleic acid synthesis, a complex is formed between a polymerase enzyme, a template nucleic acid sequence, and a priming sequence that serves as the point of initiation of the synthetic process. During synthesis, the polymerase samples nucleotide monomers from the reaction mix to determine their complementarity to the next base in the template sequence. When the sampled base is complementary to the next base, it is incorporated into the growing nascent strand. This process continues along the length of the template sequence to effectively duplicate that template. Although described in a simplified schematic fashion, the actual biochemical process of incorporation can be relatively complex. A diagrammatical representation of the incorporation biochemistry is provided in FIG. 15. This diagram is not a complete description of the mechanism of nucleotide incorporation. During the reaction process, the polymerase enzyme undergoes a series of conformational changes in the mechanism.

As shown in FIG. 15, the synthesis process begins with the binding of the primed nucleic acid template (D) to the polymerase (P) at step 2. Nucleotide (N) binding with the complex occurs at step 4. Step 6 represents the isomerization of the polymerase from the open to closed conformation. Step 8 is the chemistry step in which the nucleotide is incorporated into the growing strand. At step 10, polymerase isomerization occurs from the closed to the open position. The polyphosphate component that is cleaved upon incorporation is released from the complex at step 12. While the figure shows the release of pyrophosphate, it is understood that when a nucleotide or nucleotide-conjugate-analog is used, the component released may be different than pyrophosphate. In many cases, the systems and methods of the invention use a nucleotide-conjugate-analog having a luminescent-substrate (e.g., coelantarazine, FMNH2, or the like) on its terminal phosphate, such that the released component comprises a polyphosphate connected to a luminescent-substrate (e.g., a luminescdent-substrate-attached-leaving-group or PPᵢ—LS). With a natural nucleotide or nucleotide-conjugate-analog substrate, the polymerase then translocates on the template at step 14. After translocation, the polymerase is in the position to add another nucleotide and continue around the reaction cycle.

Suitable polymerase enzymes for use herein include DNA polymerases, which can be classified into six main groups based upon various phylogenetic relationships, e.g., with E. coil Pol I (class A), E. coli Pol II (class B), E. coil Pol III (class C), Euryarchaeotic Pol II (class D), human Pol beta (class X), and E. coil UmuC/DinB and eukaryotic RAD30/xeroderrna pigmentosum variant (class Y). For a review of nomenclature, see, e.g., Burgers et al. (2001) “Eukaryotic DNA polymerases: proposal for a revised nomenclature” J Biol Chem. 276(47):43487-90. For a review of polymerases, see, e.g., Hubscher et al. (2002) “Eukaryotic DNA Polymerases” Annual Review of Biochemistry Vol. 71: 133-163; Alba (2001) “Protein Family Review: Replicative DNA Polymerases” Genome Biology 2(1):reviews 3002.1-3002.4; and Steitz (1999) “DNA polymerases: structural diversity and common mechanisms” J Biol Chem 274:17395-17398; each of which are incorporated herein by reference in their entirety. The basic mechanisms of action for many polymerases have been determined. The sequences of literally hundreds of polymerases are publicly available, and the crystal structures for many of these have been determined, or can be inferred based upon similarity to solved crystal structures for homologous polymerases.

Many such polymerases suitable for nucleic acid sequencing are readily available. For example, human DNA Polymerase Beta is available from R&D systems. Suitable DNA polymerase for use herein, include DNA polymerase I that is available from Epicenter, GE Health Care, Invitrogen, New England Biolabs, Promega, Roche Applied Science, Sigma Aldrich and many others. The Klenow fragment of DNA Polymerase I is available in both recombinant and protease digested versions, from, e.g., Ambion, Chimerx, eEnzyme LLC, GE Health Care, Invitrogen, New England Biolabs, Promega, Roche Applied Science, Sigma Aldrich and many others. PHI.29 DNA polymerase is available from e.g., Epicentre. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of these and other sources. Other commercial DNA polymerases include PhusionhM High-Fidelity DNA Polymerase, available from New England Biolabs; GoTaq.RTM. Flexi DNA Polymerase, available from Promega; RepIiPHI.TM. .PHI.29 DNA Polymerase, available from Epicentre Biotechnologies; PfuUltra.TM. Hotstart DNA Polymerase, available from Stratagene; KOD HiFi DNA Polymerase, available from Novagen; and many others.

Available DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc. As noted, polymerases have also been modified to confer improvements in specificity, processivity, and improved retention time of labeled nucleotides in polymerase-DNA-nucleotide complexes (e.g., WO 2007/076057 POLYMERASES FOR NUCLEOTIDE ANALOGUE INCORPORATION by Hanzel et al. and WO 2008/051530 POLYMERASE ENZYMES AND REAGENTS FOR ENHANCED NUCLEIC ACID SEQUENCING by Rank et al.), to alter branch fraction and translocation (e.g., U.S. Pat. Application Ser. No. 12/584,481 filed Sep. 4, 2009, by Pranav Patel et al. entitled “ENGINEERING POLYMERASES AND REACTION CONDITIONS FOR MODIFIED INCORPORATION PROPERTIES”), to increase photostability (e.g., U.S. Pat. Application Ser. No. 12/384,110 filed Mar. 30, 2009, by Keith Bjornson et al. entitled “Enzymes Resistant to Photodamage”), and to improve surface-immobilized enzyme activities (e.g., WO 2007/075987 ACTIVE SURFACE COUPLED POLYMERASES by Hanzel et al. and WO 2007/076057 PROTEIN ENGINEERING STRATEGIES TO OPTIMIZE ACTIVITY OF SURFACE ATTACHED PROTEINS by Hanzel et al.). Any of these available polymerases can be modified in accordance with the invention to decrease branching fraction formation, improve stability of the closed polymerase-DNA complex, and/or alter reaction rate constants.

DNA polymerases that are preferred substrates for mutation to decrease branching fraction, increase closed complex stability, or alter reaction rate constants include Taq polymerases, exonuclease deficient Taq polymerases, E. coil DNA Polymerase 1, Klenow fragment, reverse transcriptases, PHI-29 related polymerases including wild type PHI-29 polymerase and derivatives of such polymerases such as exonuclease deficient forms, T7 DNA polymerase, T5 DNA polymerase, an RB69 polymerase, etc.

In addition, the polymerases can be further modified for application-specific reasons, such as to increase photostability, e.g., as taught in U.S. Pat. Application Ser. No. 12/384,110 filed Mar. 30, 2009, to improve activity of the enzyme when bound to a surface, as taught, e.g., in WO 2007/075987, and WO 2007/076057, or to include purification or handling tags as is taught in the cited references and as is common in the art. Similarly, the modified polymerases described herein can be employed in combination with other strategies to improve polymerase performance, for example, reaction conditions for controlling polymerase rate constants such as taught in U.S. Pat. Application Ser. No. 12/414,191 filed Mar. 30, 2009, and entitled “Two slow-step polymerase enzyme systems and methods,” incorporated herein by reference in its entirety for all purposes.

As used herein, the phrase “template nucleic acid” or “target template nucleic acid” refers to any suitable polynucleotide, including double-stranded DNA, single-stranded DNA, single-stranded DNA hairpins, DNA/RNA hybrids, RNAs with a recognition site for binding of the polymerizing agent, and RNA hairpins. Further, target polynucleotides suitable as template nucleic acids for use in the invention sequencing methods may be a specific portion of a genome of a cell, such as an intron, regulatory region, allele, variant or mutation; the whole genome; or any portion thereof. In other embodiments, the target polynucleotides may be mRNA, tRNA, rRNA, ribozymes, antisense RNA or RNAi. In particular embodiments, e.g., where only a single polymerase is contemplated for use to sequence a particular target, the target polynucleotide may be of any length, such as between about 10 bases up to about 100,000 bases, between about 10,000 bases up to about 90,000 bases, between about 20,000 bases up to about 80,000 bases, between about 30,000 bases up to about 70,000 bases, between about 40,000 bases up to about 60,000 bases, or longer, with a typical range being between about 10,000 - 50,000 bases. Also contemplated herein, e.g., in particular single polymerase embodiments, are target template nucleic acid lengths of between about 100 bases and 10,000 bases. Also contemplated herein, in embodiments using multiple polymerases per template nucleic acid, in addition the template nucleic acid lengths set forth above, the template nucleic acid length can be more than 100,000, between 100,000 bases and 1,000,000, between 1,000,000 bases to 1,000,000,000 bases, or more than 1,000,000,000 bases.

Accordingly, because nucleic acid sequence read-lengths can be up to the entire length of the template nucleic acid being sequenced using the invention methods, the base-pair read-lengths achieved by the invention methods are selected from the group consisting of at least: 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 60000, 70000, 80000, 90000, 100000, 200000, 300000, 400000, 500000, 600000, 700000, 800000, 900000, 1000000 (i.e., 1×10⁶), 10000000 (1×10⁷), 100000000 (1×10⁸), 1000000000 (1×10⁹), or more.

The template nucleic acids of the invention can also include unnatural nucleic acids such as PNAs, modified oligonucleotides (e.g., oligonucleotides comprising nucleotides that are not typical to biological RNA or DNA, such as 2′-O-methylated oligonucleotides), modified phosphate backbones and the like. A nucleic acid can be e.g., single-stranded or double-stranded.

As used herein, the term “primer” refers to an oligonucleotide molecule comprising any length that is sufficient to bind to the template nucleic acid and permit enzymatic extension during nucleic acid synthesis chain-elongation reaction. In particular embodiments, the primer is one continuous strand of from about 12 to about 100 nucleotides in length; more particulary is greater than or equal to: 12, 15, 20, 25, 30, 35, 40, 45, 50, 55, 60, 70, 75, 80, 85, 90, 95, or 100 nucleotides in length. In other embodiments, the primer islonger than 100 nucleotides, such as is greater than or equal to: 110, 120, 130, 140, 150, 160, 170, 180, 190, 200, 250, 300, 350, 400, 450, 500, 550, 600, 650, 700, 750, 800, 850, 900, 950, or 1,000 nucleotides in length. In particular embodiments where the invention methods are used for nucleic acid target detection, the primer is a primer-probe.

### Methods for Detecting Target Nucleic Acids

Also provided herein are methods for detecting the presence of a target nucleic acid sequence in a sample comprising:
providing an elongation mixture comprising (i) a polymerase enzyme, (ii) a luminescence enzyme, (iii) a template nucleic acid sample, (iv) a primer-probe that hybridizes to (e.g., that is complementary to) a particular target nucleic acid sequence, and (v) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has the same, or different, luminescent-substrate attached thereto, wherein the luminescent-substrate-attached-leaving-group is cleaved upon polymerase-dependent binding of a respective nucleotide-conjugate-analog to the template strand;
carrying out nucleic acid elongation synthesis such that a plurality of nucleotide-conjugate-analogs are added sequentially to the template if the primer-probe hybridizes to the target nucleic acid sequence, whereby: a) a nucleotide-conjugate-analog associates with the polymerase, b) the nucleotide-conjugate-analog is incorporated on the template strand by the polymerase when the luminescent-substrate-attached-leaving-group on that nucleotide-conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce luminescence; and
detecting light from the luminescence while nucleic acid synthesis is occurring, whereby detection of light indicates the presence of the particular target nucleic acid sequence.

In particular embodiments, the amount of target nucleic acid is quantified. In one embodment, the amount of target nucleic acid is quantified based on the intensity of the luminescence. In a particular embodiment, each type of nucleotide-conjugate-analog has the same luminescent-substrate attached thereto. In particular embodiments, a plurality of polymerase enzymes are used.

In other embodiments, one, two, three or all nucleotide-conjugate-analogs are labelled with the same luminescent-substrate analog. The reaction elongation mixture contains one or more template oligonucleotides. Upon binding of the primer-probes to the template nucleic acids and upon binding of polymerases to the primer-template complexes, DNA chain elongation reactions commence on one or more of the complexes. Each reaction generates a constant stream of cleaved luminescent substrates (e.g., PPi-LS; luminescent-substrate-attached-leaving-groups), which are fed into the luminescent reactions generating luminescent signal. In particular embodiments, the luminescent signal intensity generated is correlated to the number of primer-template pairs; and therefore is used to detect and quantify the presence of those primer-template pairs. In this particular embodiment, primer sequences are used as probe sequences to detect the presence of a specified target-complementary sequence on the template oligonucleotide. Therefore, in addition to determining the sequence, invention methods are also provided herein that allow detection and/or quantification of a particular sequence (segment) on the template oligonucleotide; similar to the goal for other molecular biology methods such as polymerase chain reaction or micro arrays. These invention target detection methods are useful in rapid detection, point of care, nucleic acid detection.

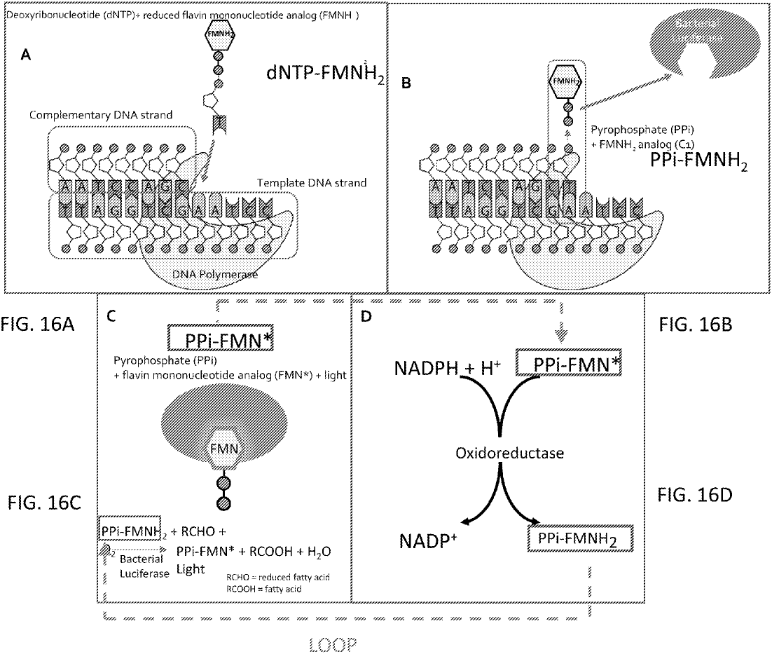

In yet another embodiment, an enzymatic loop is generated that can be used to create a continuous luminescence signal for each nucleotide (e.g., nucleotide-conjugate-analog) that is attached or incorporated into the template strand, thus amplifying the luminescence signal (see FIG. 16). With each nucleotide-conjugate-analog that is incorporated in the template nucleic acid strand, a new enzymatic loop will be generated adding to the total luminescence generated. This enzymatic loop embodiment is particularly beneficial for applications such as detection of the presence of a particular target nucleic acid sequence using the primer oligonucleotide as a probe (e.g., a primer-probe). In one embodiment, referred to herein as the oxidoreductase/Luciferase Loop, a reduced flavin mononucleotide (or an analog thereof) is attached to the terminal phosphate (dNTP-FMNH2) of one, two, three, or all four of the nucleotides. Following incorporation of a nucleotide-conjugate-analog into the template strand by polymerase, pyrophosphate attached to a reduced flavin mononucleotide analog (PPi-FMNH2) is released as a luminescence-substrate-attached-leaving-group, which then is oxidized by a bacterial luciferase generating luminescence. In the presence of oxidoreductase enzyme used in this particular embodiment, the oxidized flavin mononucleotide analog (PPi-FMN*) is reduced by oxidoreductase to PPi-FMNH2, while also converting dihydronicotinamide-adenine dinucleotide phosphate (NADPH) into the oxidized form, NADP+. This generates a luminescence reaction loop that continues as long as reduced fatty acid (RCOOH) is completely depleted in solution. In another embodiment, one can further include fatty acid reductase to further recycle reduced fatty acid by consuming ATP.

As use herein, the term “oxidoreductase/Luciferase loop” or grammatical variations thereof, refers to generally as an enzymatic loop between the oxidoreductase enzyme and luciferase (FIGS. 16C-D), whereby following the luminescent reaction of a reduced flavin mononucleotide analog (PPi-FMNH2) catalyzed by bacterial luciferase, an oxidoreductase enzyme then reduces the formed oxidized flavin mononucleotide analog (PPi-FMN*) back to the initial reduce PPi-FMNH2, also converting dihydronicotinamide-adenine dinucleotide phosphate (NADPH) into the oxidized form, NADP+. This generates a luminescence reaction loop that goes on as long as reduced fatty acid (RCOOH) is completely depleted in solution. In other embodiments, fatty acid reductase can be added the reaction mixture to further recycle reduced fatty acid by consuming ATP. This oxidoreductase/Luciferase enzymatic loop will generate successive signals from the FMNH2-attached-pyrophosphate leaving group, and thereby serve as an amplification mechanism for the luciferase signal produced from the enzymatic incorporation of the most recent nucleotide.

As set forth herein, this pyrophosphate (PPi-FMN*) from FIG. 16C can loop numerous times back via the reaction set forth in FIG. 16D in the oxidoreductase/Luciferase Amplification Loop. The number of times pyrophosphate (PPi-FMN*) can be looped back to amplify the respective luminescence signal for each nucleotide-analog-conjugate (dNTP) incorporation event into the elongating sequence can be selected from the group consisting of at least: 5, 10, 20, 30, 40, 50, 60, 70, 80, 90, 100, 150, 200, 250, 300, 350, 400, 450, 500, 550, 600, 650, 700, 750, 800, 850, 900, 950, 1000, 10000, 20000, 30000, 40000, 50000, 60000, 70000, 80000, 90000, 100000, 200000, 300000, 400000, 500000, 600000, 700000, 800000, 900000, and at least 1000000 times.

As used herein, the term “primer-probe” refers to a primer that can initiate chain elongation that also functions as a probe to identify a particular target nucleic acid sequence, preferably from among a sample of unknown nucleic acids being interrogated. Since there is no temperature cycling and denaturation, and hybridization cycles do not exist such as for PCR, there is a great deal of flexibility in the probe design in terms of length and sequence that can be used in the invention methods. With the invention methods provided herein, designing one oligonucleotide probe (e.g., a primer-probe) is sufficient, instead of using 2 primers as is required for PCR. The length of the primer-probe can be any size, so long as it accurately binds to its respective target nucleic acid sequence from among the template nucleic acid sample. For example, in addition to the lengths set forth above for primers, other suitable ranges of primer-probe lengths for use herein can be selected from the group consisting of: 20-100, 20-90, 20-80, 20-70, 20-60, 20-50, 20-40, 20-30, 5-100, 10-100, 30-100, 40-100, 50-100, 60-100, 70-100, 80-100, 90-100, 15-150, 10-200, 5-300, 20-200, 20-300, 20-400, 20-500, 20-600, 20-700, 20-800, 20-900, 20-1000, at least 5, at least 10, at least 15, at least 20, at least 30, at least 40, at least 50, at least 60, at least 70, at least 80, at least 90, at least 100, at least 150, at least 200, at least 300, at least 400, at least 500, at least 600, at least 700, at least 800, at least 900 at least 1000 nucleotide bases.

Other ranges of primer-probe lengths suitable for use herein can be selected from the group consisting of: 5-1000 bases, 10-950, 15-900, 20-800, 25-700, 30-600, 35-500, 40-400, 50-300, 25-250, 25-200, 25-150, 25-100, 25-90, 25-80, 25-70, 25-60, 25-50 base in length. In other embodiments, the primer-probe is in the range of 20-100 bases. In other embodiments, those of skill in the art can select a longer nucleotide sequence for the primer-probe length from the group consisting of: 25, 30, 40, 45, 50, 55, 60, 65, 70, 80, 85, 90, 95, 100, 110, 120, 130, 140, 150, 160, 170, 180, 190, and 200 bases or more to increase specificity. In other embodiments, as with PCR, a probe length about 20 bases is also contemplated for use herein.

### Nucleotide-Conjugate-Analogs

Also provided herein are nucleotide-conjugate-analogs, comprising a deoxyribonucleotide (dNTP), or analog thereof; and luminescent-substrate attached thereto. As used herein, the phrase “nucleotide-conjugate-analog” (also referred to herein as “luminescent-substrate-nucleotide conjugates”) refers to any nucleotides modified with a luminescent-substrate that can be used in DNA synthesis (e.g., modified dNTPs such dATP, dTTP, dGTP, dCTP and dUTP). In some embodiments, the nucleotides within the nucleotide-conjugate-analogs are modified nucleotide analogs. The nucleotide analogs for use in the invention can be any suitable nucleotide analog that is capable of being a substrate for the polymerase and for the selective cleaving activity. It has been shown that nucleotides can be modified and still used as substrates for polymerases and other enzymes. Where a variant of a nucleotide analog is contemplated, the compatibility of the nucleotide analog with the polymerase or with another enzyme activity such as exonuclease activity can be determined by activity assays. The carrying out of activity assays is straightforward and well known in the art.

In particular embodiments of the invention methods set forth herein, the invention nucleotide-conjugate-analog can be, for example, a nucleoside polyphosphate having three or more phosphates in its polyphosphate chain with a luminescent substrate attached to the portion of the polyphosphate chain that is cleaved upon incorporation into the growing strand; which results in the luminescent-substrate-attached-leaving-group. The polyphosphate can be a pure polyphosphate, e.g. —O—PO3— or a pyrophosphate (e.g., PPi), or the polyphosphate can include substitutions. Additional details regarding analogs and methods of making such analogs can be found in U.S. Pats. 7,405,281; 9,464,107, and the like; incorporated herein by reference in its entirety for all purposes.

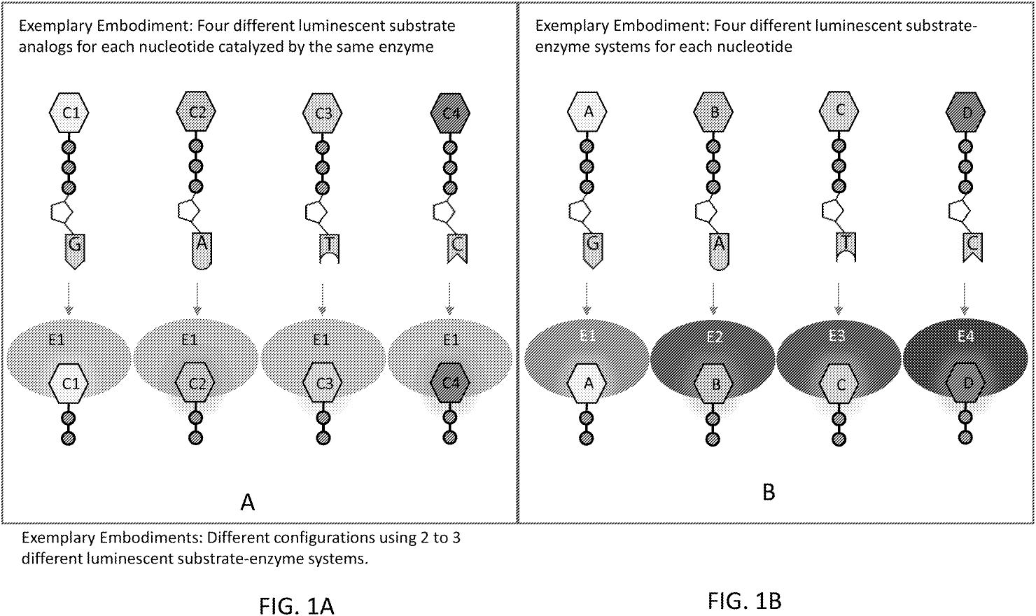

In other embodiments of the invention, to form a nucleotide-conjugate-analog, a nucleotide or analog thereof, is modified by adding a luminescent-substrate (e.g., coelenterazine, FMNH2, and the like) to a terminal phosphate (see, e.g, Yarbrough et al., J. Biol. Chem., 254: 12069-12073, 1979; incorporated herein by reference in its entirety for all purposes), such that when the PPi luminescent-substrate-attached-leaving-group (e.g., PPi-LS, PPi-C; PPi-FMNH2, and the like) is generated by the polymerase when the luminescent-substrate nucleotide conjugate is incorporated into the template strand, the luminescent-substrate-attached-pyrophosphate (or luminescent-substrate-attached-leaving-group) is able to be combined with the respective luciferase (see FIGS. 1-3). There are five types of dNTPs, namely deoxyadenosine triphosphate (dATP), deoxyguanosine triphosphate (dGTP), deoxycytidine triphosphate (dCTP), deoxythymidine triphosphate (dTTP), and deoxyuradine triphosphate (dUTP). Four or five of these dNTPs are used in the template directed nucleic acid synthesis reaction to identify (i.e., call) its complement (e.g., adenine, guanine, cytosine, or thymine) in the template nucleic acid strand, thereby sequencing the template nucleic acid strand. Instead of dATP, dATPαS might be used as a substitute for the dATP as it acts as a substrate for DNA polymerase but not for luciferase.

Each modified nucleotide-conjugate-analog generates a unique luminescent signal (e.g., wavelengths of 411, 417, 428, 440, 484, 509 nm, and the like) from the attached luminescent substrate while they are being attached to the complementary strand by the polymerase enzyme. In one embodiment, the unique luminescence signal is a wavelength selected from the range 250 nm - 750 nm. In another embodiment, the unique luminescent signal can be a wavelength selected from the group consisting of: 411, 417, 428, 440, 484, and 509 nm.

Also provided herein is a chain-elongation set of nucleotide-conjugate-analogs comprising at least 4 distinct a deoxyribonucleotides (dNTPs), such that the chain-elongation set can be incorporated into template directed synthesis of a growing nucleic acid strand. Either dTTP or dUTP or any combination of both can be used in a nucleic acid synthesis chain elongation reaction to call (i.e., identify) the complementary adenine (ATP) in the sequence. If both modified dTTP and dUTP analogs are used in the reaction, they can each have the same luminescent substrate attached thereto producing the same wavelength signal; or each can have a discreet luminescent substrate attached thereto.

In preferred embodiments of the invention methods disclosed herein, each respective dNTP, or analog thereof, is modified using a different, unique luminescent substrate (e.g., coelenteerazine analogs, FMNH2 analogs, and the like) relative to the other dNTPs, such that each time a polymerase incorporates a modified deoxyribonuleoside triphosphate (dNTP) nucleotide-conjugate-analog to the strand complementary to the template DNA, a luminescent signal specific to the class or type of the respective nucleotide (e.g., unique signals for each of dATP, dATPαS, dTTP, dGTP and dCTP, or other modified nucleotides well-known in the art) attached is generated. Other modified nucleotides contemplated for use herein are well-known in the art such as those described in Jordheim et al., Advances in the development of nucleoside and nucleotide analogues for cancer and viral diseases, Nat. Rev. Drug Discov. (2013) 12: 447-464; and Guo et al. Four-color DNA sequencing with 3′-O-modified nucleotide reversible terminators and chemically cleavable fluorescent dideoxynucleotides, Proc. Natl. Acad. Sci. U.S.A. (2008) 105:9145-9150, and the like (each of which are incorporated by reference herein in their entirety).

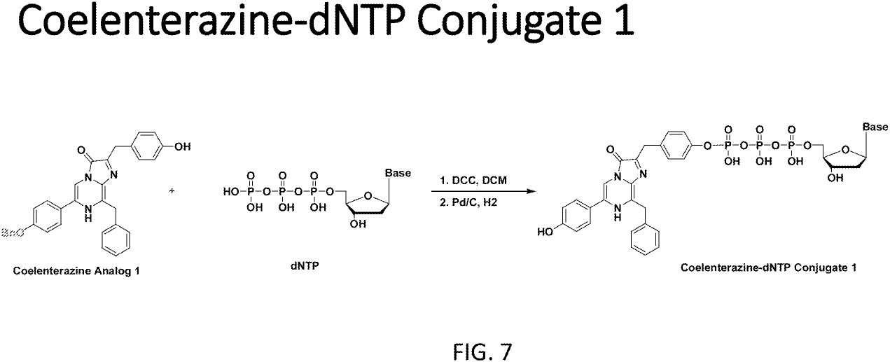

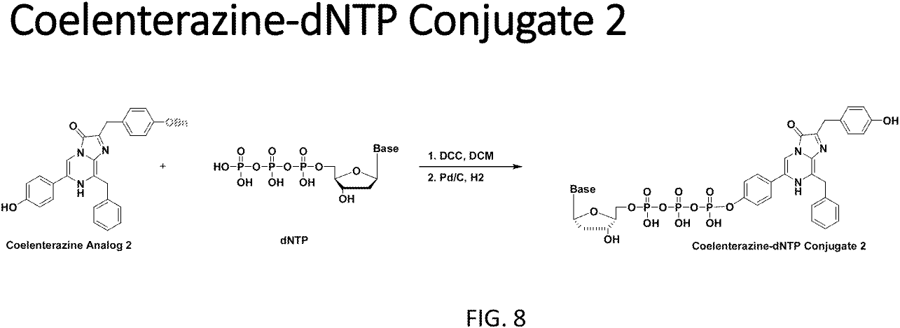

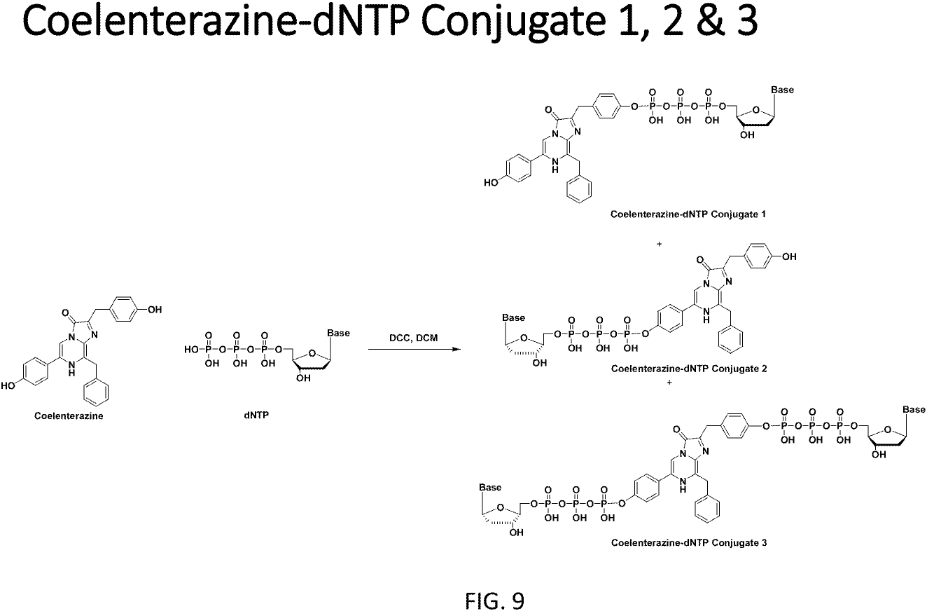

In particular embodiments, exemplary nucleotide-conjugate-analogs, also referred to herein as “luminescent-substrate attached-dNTPs,” for use herein include: Coelenterazine-dNTP Conjugate 1 (FIG. 7); Coelentarazine-dNTP Conjugate 2 (FIG. 8); Coelentarazine-dNTP Conjugate 3 (FIG. 9); and the like.

In yet other embodiments, dATPαS, dGTPαS, dCTPαS, dTTPαS are used in place of dATP, dGTP, dCTP and dTTP, which is contemplated herein to reduce the non-specific interaction of nucleotides with enzymes other than polymerase (e.g., luciferase).

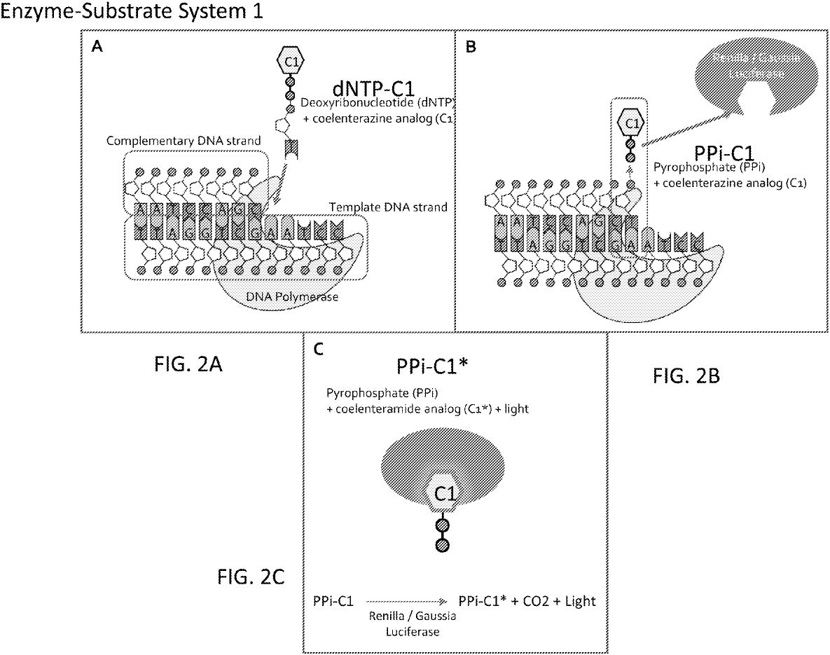

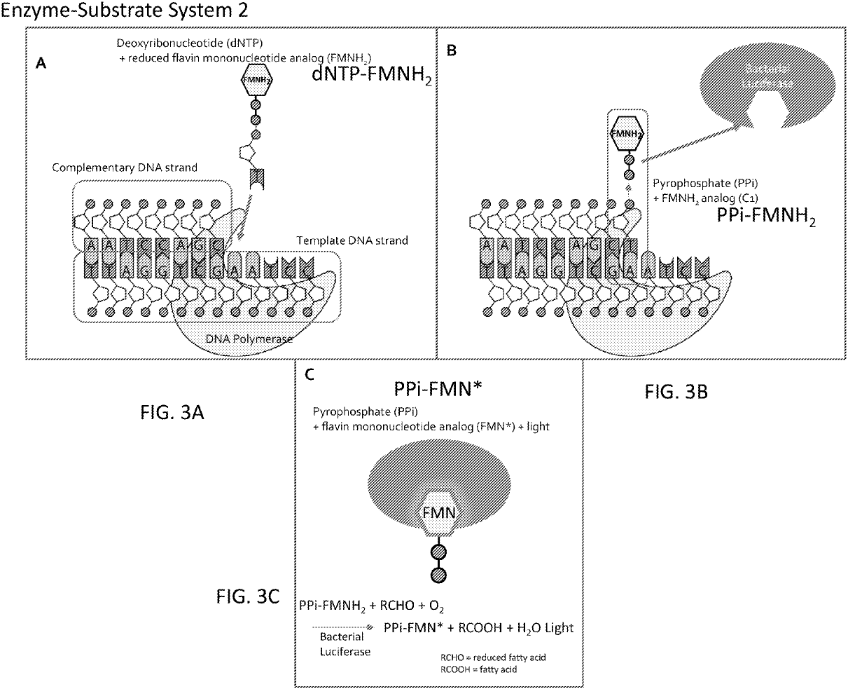

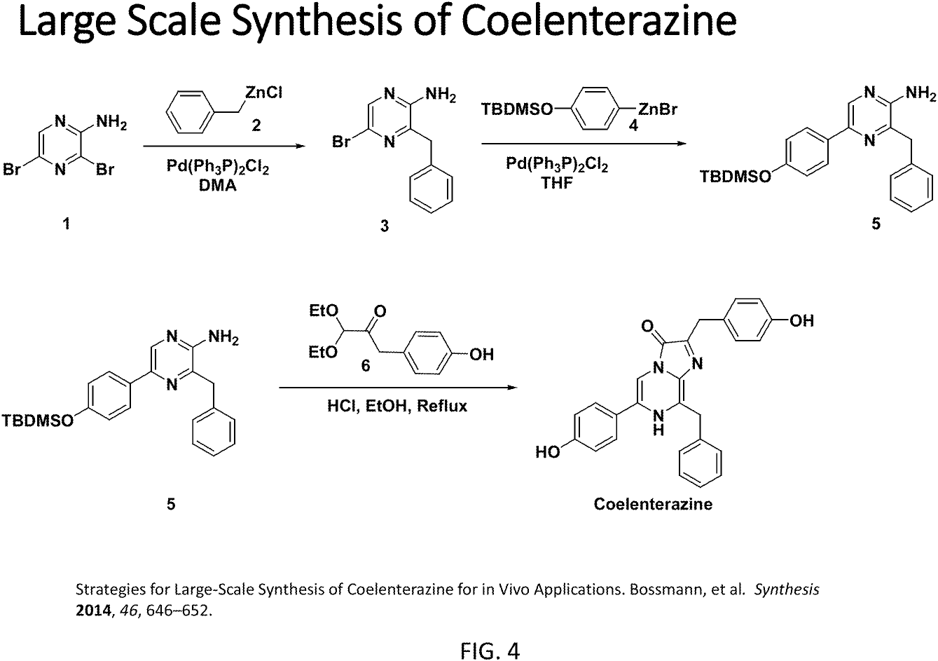

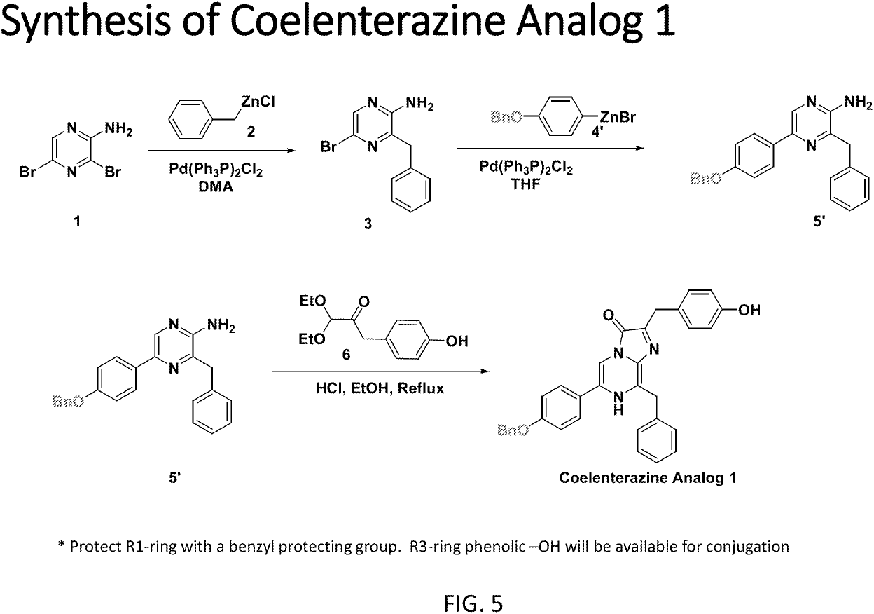

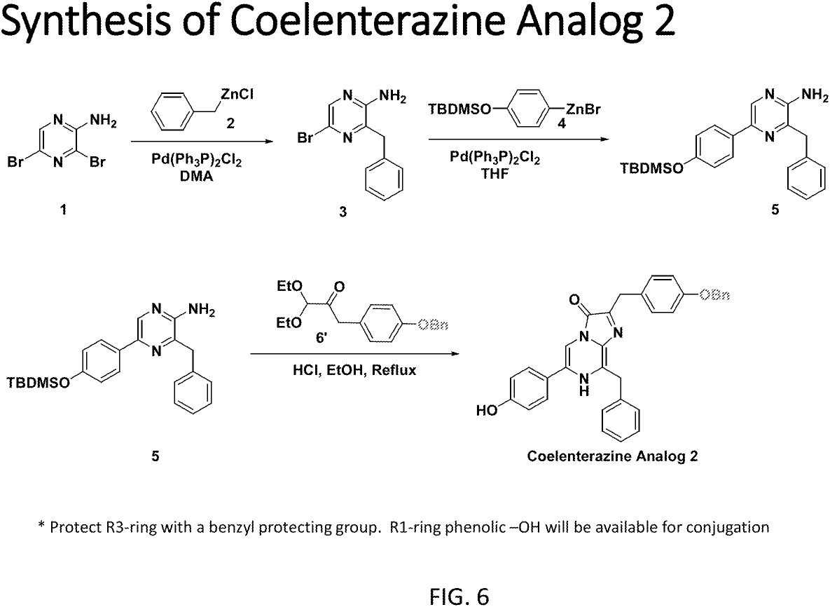

Each nucleotide-conjugate-analog effectively generates a unique luminescent signal or spectra (e.g., in red, yellow, green, or blue, and the like) while they are being attached to the complementary strand by the polymerase enzyme. Upon the completion of attachment of the nucleotide-conjugate-analog to the 3′ moiety of the previously attached nucleotide-conjugate-analog, as a result of the subsequent luminescence reactions the luminescence signal (spectra) generated by the luminescent-substrate-attached-pyrophosphate leaving group (e.g., PPi + LS, PPi-C, PPi-FMH2, and the like) is detected by an appropriate luminescence sensor and/or detection device during the discreet and limited period of the respective luminescence reactions (FIG. 2C and FIG. 3C).

Using the invention concatenated 2-Enzyme system and methods provided herein, a particular signal indicating the particular type of nucleotide will be generated only during the specific interaction of the nucleotide with the polymerase-Luciferase reactions. The pre- and post- polymerase interaction states will be similar; and the signal will “change” during the interaction with the polymerase. For example, in one embodiment described herein:

1- Initially because there is no external light excitation, there is either none or very low background luminescence.

2- During the polymerase-luciferase interaction of the invention methods, a specific type of luminescence is generated.

3- After the respective luminescence reaction ceases the luminescent-substrate-attached-pyrophosphate signal (PPi + LS) goes back to the initial state.

As used herein, the phrase “luminescent-substrate-attached-leaving-group” refers to the polyphosphate chain having a luminescence-substrate, or the like, attached therein, that is released from a respective dNTP when and/or upon cleavage by the invention 2 enzyme polymerase-luciferase reaction during the incorporation of the respective dNTP into the template nucleic acid strand. In a particular embodiment herein, the polyphosphate is a luminescent pyrophosphate (PPi + LS) that is cleaved from dNTP (FIGS. 2B and 3B), and then subsequently enters the luciferase reaction (FIGS. 2C and 3C) for subsequent luminescence detection prior to the termination of the respective, discreet, limited-period luminescence reaction as set forth herein (see FIGS. 2C and 3C).

The reaction conditions used can also influence the relative rates of the various reactions. Thus, controlling the reaction conditions can be useful in ensuring that the sequencing method is successful at calling the bases within the template at a high rate. The reaction conditions include, e.g., the type and concentration of buffer, the pH of the reaction, the temperature, the type and concentration of salts, the presence of particular additives which influence the kinetics of the enzyme, and the type, concentration, and relative amounts of various cofactors, including metal cofactors. Manipulation of reaction conditions to achieve or enhance the two slow-step behavior of polymerases is described in detail in U.S. Pat. 8,133,672, incorporated herein by reference.

Enzymatic reactions are often run in the presence of a buffer, which is used, in part, to control the pH of the reaction mixture. The type of buffer can in some cases influence the kinetics of the polymerase reaction in a way that can lead to two slow-step kinetics, when such kinetics are desired. For example, in some cases, use of IRIS as buffer is useful for obtaining a two slow-step reaction. Suitable buffers include, for example, TAPS (3-{[tris(hydroxymethyl)methyl]amino}propanesulfonic acid), Bicine (N,N-bis(2-hydroxyethyl)glycine), IRIS (tris(hydroxymethyl)methylamine), ACES (N-(2-Acetamido)-2-aminoethanesulfonic acid), Tricine (N-tris(hydroxymethyl)methylglycine), HEPES 4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid).

The pH of the reaction can influence the kinetics of the polymerase reaction, and can be used as one of the polymerase reaction conditions to obtain a reaction exhibiting two slow-step kinetics. The pH can be adjusted to a value that produces a two slow-step reaction mechanism. The pH is generally between about 6 and about 9. In some embodiments, the pH is between about 6.5 and about 8.0. In other embodiments, the pH is between about 6.5 and 7.5. In particular embodiments, the pH is selected from about 6.5, 6.6, 6.7, 6.8, 6.9, 7.0, 7.1, 7.2, 7.3, 7.4, or 7.5.

The temperature of the reaction can be adjusted to ensure that the relative rates of the reactions are occurring in the appropriate range. The reaction temperature may depend upon the type of polymerase or selective cleaving activity employed. The temperatures used herein are also contemplated to manipulate and control the hydrogen bonding between two bases as well as the bases’ interaction with the water in the reaction mixture, thereby controlling the solubility of the reaction components.

In some embodiments, additives, such as magnesium, Coenzyme A, and the like, can be added to the reaction mixture that will influence the kinetics of the reaction. In some cases, the additives can interact with the active site of the enzyme, acting for example as competitive inhibitors. In some cases, additives can interact with portions of the enzyme away from the active site in a manner that will influence the kinetics of the reaction. Additives that can influence the kinetics include, for example, competitive but otherwise unreactive substrates or inhibitors in analytical reactions to modulate the rate of reaction as described in U.S. Utility Pat. 8,252,911, the full disclosure of which is incorporated herein by reference in its entirety for all purposes.

As another example, an isotope such as deuterium can be added to influence the rate of one or more step in the polymerase reaction. In some cases, deuterium can be used to slow one or more steps in the polymerase reaction due to the deuterium isotope effect. By altering the kinetics of steps of the polymerase reaction, in some instances two slow step kinetics, as described herein, can be achieved. The deuterium isotope effect can be used, for example, to control the rate of incorporation of nucleotide, e.g., by slowing the incorporation rate. Isotopes other than deuterium can also be employed, for example, isotopes of carbon (e.g. ¹³C), nitrogen, oxygen, sulfur, or phosphorous.

As yet another example, additives that can be used to control the kinetics of the polymerase reaction include the addition of organic solvents. The solvent additives are generally water soluble organic solvents. The solvents need not be soluble at all concentrations, but are generally soluble at the amounts used to control the kinetics of the polymerase reaction. While not being bound by theory, it is believed that the solvents can influence the three dimensional conformation of the polymerase enzyme which can affect the rates of the various steps in the polymerase reaction. For example, the solvents can affect steps involving conformational changes such as the isomerization steps. Added solvents can also affect, and in some cases slow, the translocation step. In some cases, the solvents act by influencing hydrogen bonding interactions.

The water miscible organic solvents that can be used to control the rates of one or more steps of the polymerase reaction in single molecule sequencing include, e.g., alcohols, amines, amides, nitriles, sulfoxides, ethers, and esters and small molecules having more than one of these functional groups. Exemplary solvents include alcohols such as methanol, ethanol, propanol, isopropanol, glycerol, and small alcohols. The alcohols can have one, two, three, or more alcohol groups. Exemplary solvents also include small molecule ethers such as tetrahydrofuran (THF) and dioxane, dimethylacetamide (DMA), dimethylsulfoxide (DMSO), dimethylformamide (DMF), and acetonitrile.

The water miscible organic solvent can be present in any amount sufficient to control the kinetics of the polymerase reaction. The solvents are generally added in an amount less than 40% of the solvent weight by weight or volume by volume. In some embodiments the solvents are added between about 0.1% and 30%, between about 1% and about 20%, between about 2% and about 15%, and between about 5% and 12%. The effective amount for controlling the kinetics can be determined by the methods described herein and those known in the art.

Another aspect of controlling the polymerase reaction conditions relates to the selection of the type, level, and relative amounts of cofactors. For example, during the course of the polymerase reaction, divalent metal co-factors, such as magnesium or manganese, will interact with the enzyme-substrate complex, playing a structural role in the definition of the active site. For a discussion of metal co-factor interactions in polymerase reactions, see, for example, Arndt, et al., Biochemistry (2001) 40:5368-5375. Suitable conditions include those described in U.S. Pat. 8,257,954, incorporated herein by reference in its entirety for all purposes.

In a particular embodiment of the invention methods, the rate and fidelity of the polymerase reaction is controlled by adjusting the concentrations of the dNTP nucleotide-conjugate-analogs such that the polymerase operates in near ideal conditions in terms of parameters such as substrate concentration, amount of optical excitation, level of chemical modification. Therefore, the polymerase enzyme is contemplated herein to reach its maximum read-lengths, e.g., approximately in the tens of thousands of base pairs, similar to the DNA synthesis lengths achieved in natural settings. This reduces device complexity and increases enzymatic sensitivity and specificity leading to low error-rates and thus low coverage. This not only reduces the cost of the device as well as cost per genome, but also makes applications such as single-nucleotide polymerism detection, structural variation, and genome assembly possible in a very compact system.

### Method of Achieving Long Read-Lengths in Single Molecule Reactions

The ability to achieve long read-lengths has been an elusive goal for existing sequencing methods. Modern sequencing approaches are limited in their ability to achieve long read-lengths. In particular, for single molecule sequencing methods this limitation comes from the relative affinity of the polymerase to the template DNA. During the sequencing reaction, polymerase will eventually fall from the template DNA thereby terminating the dNTP chain elongation reaction at that respective read length. For example with typical sequencing technologies, there is one template and one polymerase per cell. For these single polymerase sequencing reactions, when the single polymerase dissociates from the template (falls away), the length of that particular read terminates, typically at relatively short read lengths corresponding to what is believed to be about 700 base pairs (bp).

Provided herein, in accordance with the present invention, are methods of sequencing a template nucleic acid, comprising:
providing a sequencing mixture as described herein comprising: a target template nucleic acid and a primer, a plurality of types of nucleotide-conjugate-analogs, and plurality of polymerase enzymes;
carrying out nucleic acid synthesis such that a plurality of nucleotide-conjugate-analogs are added sequentially to the template; and
detecting a respective nucleotide-conjugate-analog while nucleic acid synthesis is occurring, to determine a sequence of the template nucleic acid.

As used herein, the phrase “plurality of polymerase enzymes,” “plurality of polymerases” or grammatical variations thereof, refers the number of polymerase enzymes per nucleic acid template to be sequenced, used in a single sequencing reaction mixture. The quantity of polymerases in the “plurality of polymerase enzymes” for each template strand to be sequenced, can be selected from the group consisting of at least: 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 10000, 20000, 30000, 40000, 50000, 60000, 70000, 80000, 90000, 100000, 200000, 300000, 400000, 500000, 600000, 700000, 800000, 900000, and at least 1000000 polymerase enzymes, for each template strand to be sequenced. In other embodiments of continuously sequencing a target nucleic acid template, the ratio of polymerase to template is selected from the group consisting of at least 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 10000:1, 20000:1, 30000:1, 40000:1, 50000:1, 60000:1, 70000:1, 80000:1, 90000:1, 100000:1, 200000:1, 300000:1, 400000:1, 500000:1, 600000:1, 700000:1, 800000:1, 900000:1, and at least 1000000:1. The polymerases in the plurality can be a homogeneous collection of the same type of polymerase, or can be a heterogeneous collection of 2 or more different types of polymerases, e.g. 3, 4, 5, 6, 7, 8, 9, 10, 20, 30, 40, 50 up to 100 or more different polymerases in the plurality.

In particular embodiments, the single sequencing or target detection reaction mixture has only one (a single) target template nucleic acid to be sequenced therein, with one or more primers. In other embodiments, the single sequencing or target detection reaction mixture has more than one, or multiple, or a plurality of target template nucleic acid to be sequenced therein, with a plurality of primers. In a particular embodiment, one target template nucleic acid is provided in an individual optical confinement.

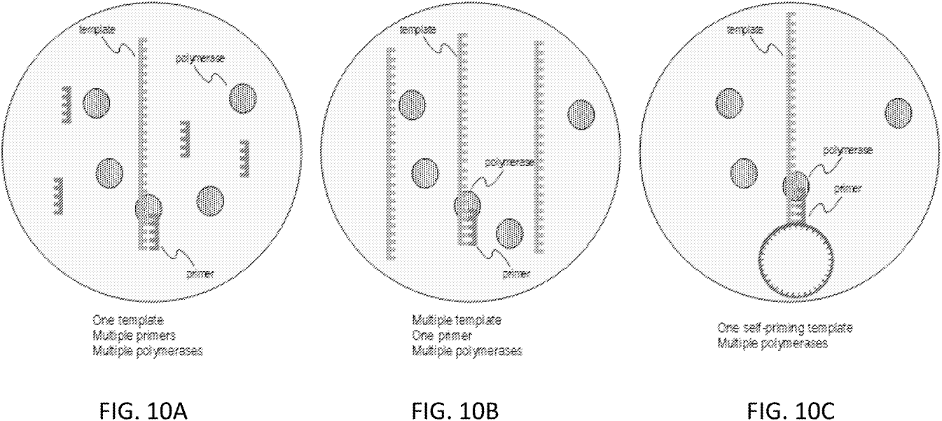

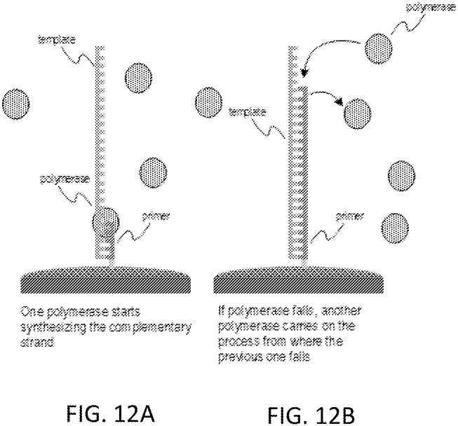

In some embodiments of the invention LASH sequencing methods, the enzyme concatenate is provided in a particular individual confinement (e.g., a droplet, or the like), such that there is only one template target nucleic acid in the confinement area, while there is a plurality (e.g., many) of polymerase enzymes and a corresponding plurality of the other enzymes forming the concatenate (FIG. 10). In this embodiment, when a polymerase enzyme drops off (dissociates) from the target template nucleic acid (FIG. 12B), one of the many plurality of the other polymerases confined to the particular target nucleic acid template area, advantageously and relatively immediately commences its chain elongation at the location on the template where the previous polymerase left off or dissociated (FIG. 12B). In other words, the sequencing chain elongation occurs with a first polymerase enzyme until it gives way and dissociates from the template nucleic acid, then the sequencing chain elongation reaction continues with a second polymerase (different from the first) until it gives way and dissociates from the template nucleic acid, then the sequencing chain elongation reaction continues with a third polymerase (different from the second pol; which could be the first pol or another of the plurality of pols in the particular sequencing reaction) until it gives way and dissociates from the template nucleic acid, and so on. Those of skill in the art will readily understand that using this approach, the target nucleic acid template in continuously being sequenced, so long as the sequencing reaction is being run. Those of skill in the art will also readily understand that when using the substantially continuous method of sequencing disclosed herein, its read length is only limited by the length of the target nucleic and/or the physical size of the reaction confinement area used for the respective chain elongation reaction.

Accordingly, provided herein is a method of continuously sequencing a target nucleic acid template. In this embodiment, as used herein “continuity,” “continuously sequencing a target nucleic acid template,” or “substantially continuously sequencing a target nucleic acid template,” does not mean that a single polymerase is able to continuously sequence a particular target nucleic acids for the entire long read lengths, but rather means that the plurality of polymerase enzymes in the reaction area of the target nucleic acid template, taken together between them, are able to continuously sequence a particular target, by virtue of that plurality of polymerase enzymes continuously having numerous polymerases available to take over dNTP chain elongation at the next nucleotide from where the previous polymerase dissociated from the particular target nucleic acid template.

In particular embodiments of invention continuous LASH sequencing methods, especially where a plurality of polymerase are used to sequence a single target template nucleic acid, the overall read length is only limited by the length of target template nucleic acid that is provided to a particular reaction confinement area. For example, the overall read lengths contemplated herein that can be achieved by using a plurality of polymerases on a single target nucleic acid template, are up to the lengths of entire chromosomes, e.g., 50 million up to about 300 million base pairs (e.g, 300 Mbp), and the like. In other certain embodiments contemplated herein, read lengths achieved by the invention sequencing methods can be selected from the group consisting of at least: 200 bp, 300 bp, 400 bp, 500 bp, 600 bp, 700 bp, 80 bp, 900 bp, 1000 bp (i.e., 1 kbp), 5 kbp 10 kbp, 20 kbp, 30 kbp, 40 kbp, 50 kbp, 100 kbp, 200 kbp, 300 kbp, 400 kbp, 500 kbp, 600 kbp, 700 kbp, 800 kbp, 900 kbp, 1000 kbp (1 Mbp), 5 Mbp, 10 Mbp, 20 Mbp, 50 Mbp, 75 Mbp, 100 Mbp, 200 Mbp, 300 Mbp, 400 Mbp, 500 Mbp, 600 Mbp, 700 Mbp, 800 Mbp, 900 Mpb, 1000 Mbp.

In yet further embodiments as set forth above, because nucleic acid sequence read-lengths can be up to the entire length of the template nucleic acid being sequenced using the invention methods, the base-pair read-lengths achieved by the invention methods can be selected from the group consisting of at least: 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 60000, 70000, 80000, 90000, 100000, 200000, 300000, 400000, 500000, 600000, 700000, 800000, 900000, 1000000 (i.e., 1×10⁶), 10000000 (1×10⁷), 100000000 (1×10⁸), 1000000000 (1×10⁹), or more.

Because of the substantially continuous sequencing of the target template nucleic acid by plurality of polymerases, the reaction is not limited by a single enzyme’s ability to achieve a particular read length. This permits the use of enzymes with higher specificity and low error rates in the invention methods. In accordance with particular embodiments of the invention LASH methods of sequencing, it is contemplated herein that using one template, and more than one polymerase (i.e., a plurality) can achieve infinitely long read-lengths. As set forth herein, as one polymerase falls off the target template nucleic acid, another polymerase will continue from where the previous polymerase left off, which advantageously alters the way the polymerase can be selected or optimized to perform in the invention LASH methods of sequencing. For this reason, one of skill in the art can select a polymerase with a very low error rate, even though that polymerase may also have a relatively short read length. This provides an advantage for this particular embodiment, in that the polymerase selected for use in the invention sequencing methods does not require both long read length and specificity.

The invention includes systems for sequencing of nucleic acid templates. The systems provide for concurrently sequencing a plurality of nucleic acid templates. The system can incorporate all of the reagents and methods described herein, and provides the instrumentation required for containing the sample, illuminating the sample with excitation light from the luminescence reactions, detecting light emitted from the sample during sequencing to produce intensity versus time data from the luminescent-substrate-attached-leaving-groups (e.g, PPi-Cl, PPi-FMNH2, or the like) cleaved from the nucleotide-conjugate-analogs as the respective dNTPs are incorporated by the polymerase onto its cognate template nucleic acid; and from the respective luminescent-substrate-attached-leaving-groups, e.g., PPi-Cl or PPi-FMNH2, or the like, determining the sequence of a template using the sequential intensity versus time data.

As used herein, the phrase “detecting light” refers to well-known methods for detecting, for example, luminescence emitted from luminescent-substrates when such luminescent-substrate-leaving-groups are in their excitation state emitting their respective signal.

In one embodiment, the system for sequencing generally comprises a substrate having a plurality of single polymerase enzymes, single templates, or single primers within, for example, a unique droplet, or the like. In the case of highly processive enzyme polymerase reactions, each comprising a polymerase enzyme, a nucleic acid template, and a primer are uniquely confined such that their signals can be assigned to the respective nucleotide as gene synthesis occurs. In other embodiments provided herein a plurality of polymerase enzymes are used with a single templates and/or a single primer, within, for example, a unique confinement, droplet, or the like. The sequencing reagents generally include two or more types of nucleotide-conjugate-analogs, preferably four nucleotide-conjugate-analogs corresponding dATP, dTTP, dAGP and dCTP, each nucleotide-conjugate-analog labeled with a different luminescent-substrate label. The polymerase sequentially adds nucleotides or nucleotide-conjugate-analogs to the growing strand, which extends from the primer. Each added nucleotide or nucleotide-conjugate-analog is complementary to the corresponding base on the template nucleic acid, such that the portion of the growing strand that is produced is complementary to the template.

The system comprises luminescence reagents (e.g., luciferase and the respective luminescent-substrate) for illuminating the luminescent-substrate-attached-leaving-groups from the respective dNTPs as they are incorporated into the template strand undergoing the luminescence reaction as set forth in FIG. 2 and FIG. 3. The luminescence reaction illuminates the respective luminescent-substrate-attached-leaving-groups in a wavelength range that corresponds to a respective dNTP. As set forth herein, the luminescent-substrate can be selected from the group consisting of: colentarazine or an analog thereof; FMNH2 or an analog thereof; luminol, isoluminol, acridinium, dioxetanes, peroxyozalic, and their derivatives thereof.

The system further comprises detection optics for observing signals from the luminescent-substrate-attached-leaving-groups cleaved from the respective nucleotide-conjugate-analog during the polymerase enzyme mediated addition to the template strand. The detection optics observe a plurality of single molecule polymerase sequencing reactions concurrently, observing the nucleotide or nucleotide-conjugate-analog additions for each of them via the luminescent-substrate-attached-leaving-groups (e.g., PPᵢ-Cl or PPi-FMNH2) that is ultimately cleaved in the invention concatenated 2 enzyme (Polymerase-Luciferase) system. For each of the observed single molecule polymerase sequencing reactions, the detection optics concurrently observe the signals from each of the luminescent-substrate-attached-leaving-groups that are indicative of the respective luminescent-substrate that is excited by the respective luminescence reaction corresponding to a respective dNTP, until each discreet and limited period signal ceases due to the decay and termination of the luminescent signal from the respective luminescence reaction.

The system also comprises a computer configured to determine the type of nucleotide-conjugate-analog that is added to the growing strand using the observed signal from the respective luminescent-substrate-attached-leaving-group; whereby observed signals from the luminescent-substrate-attached-leaving-groups are used to indicate whether a type of nucleotide or nucleotide-conjugate-analog is incorporated into the growing strand. The computer generally receives information regarding the observed signals from the detection optics in the form of signal data. The computer stores, processes, and interprets the signal data, using the signal data in order to produce a sequence of base calls. The base calls represent the computers estimate of the sequence of the template from the signal data received combined with other information given to the computer to assist in the sequence determination.

Optical detections systems which can be used with the present invention are described, for example in U.S. Pats. 8,802,424; 7,714,303; and 7,820,983, each of which are incorporated herein by reference in their entirety for all purposes.

Computers for use in carrying out the processes of the invention can range from personal computers such as PC or Macintosh.RTM. type computers running Intel Pentium or DuoCore processors, to workstations, laboratory equipment, or high speed servers, running UNIX, LINUX, Windows.RTM., or other systems, Logic processing of the invention may be performed entirely by general purposes logic processors (such as CPU’s) executing software and/or firmware logic instructions; or entirely by special purposes logic processing circuits (such as ASICs) incorporated into laboratory or diagnostic systems or camera systems which may also include software or firmware elements; or by a combination of general purpose and special purpose logic circuits. Data formats for the signal data may comprise any convenient format, including digital image based data formats, such as JPEG, GIF, BMP, TIFF, or other sequencing specific formats including “fastq” or the “qseq” format (Illumina); while video based formats, such as avi, mpeg, mov, rmv, or other video formats may be employed. The software processes of the invention may generally be programmed in a variety of programming languages including, e.g., Matlab, C, C++, C#, NET, Visual Basic, Python, JAVA, CGI, and the like.

In some embodiments of the methods and systems of the invention, optical confinements are used to enhance the ability to concurrently observe multiple single molecule polymerase sequencing reactions simultaneously. In general, optical confinements are disposed upon a substrate and used to provide electromagnetic radiation to or derive such radiation from only very small spaces or volumes. Such optical confinements may comprise structural confinements, e.g., wells, recesses, conduits, or the like, or they may comprise optical processes in conjunction with other components, to provide detection or derive emitted radiation from only very small volumes. Examples of such optical confinements include systems that utilize, e.g., total internal reflection (TIR) based optical systems whereby light is directed through a transparent portion of the substrate at an angle that yields total internal reflection within the substrate.

In a particular embodiment, a preferred optical confinement is a micro-droplet (e.g., water-in-oil emulsion, and the like) which can contain and individual sequencing reaction set forth herein. For example, the sequencing mixture reaction ingredients can be split in a way that each micro-droplet contains one polymerase-luciferase set of enzymes and related reagents and one template nucleic acid whereby each signal detection unit is focused on a single micro-droplet. It is contemplated herein that each micro-droplet is a single molecule reaction cell containing individual single molecule sequencing reactions. The micro-droplet reaction cell is also advantageously useful in the invention sequencing methods to act as micro-lenses to focus light on the respective signal detection unit.

The substrates of the invention are generally rigid, and often planar, but need not be either. Where the substrate comprises an array of optical confinements, the substrate will generally be of a size and shape that can interface with optical instrumentation to allow for the illumination and for the measurement of light from the optical confinements. Typically, the substrate will also be configured to be held in contact with liquid media, for instance containing reagents and substrates and/or labeled components, such as the nucleotide-conjugate-analogs, for optical measurements.

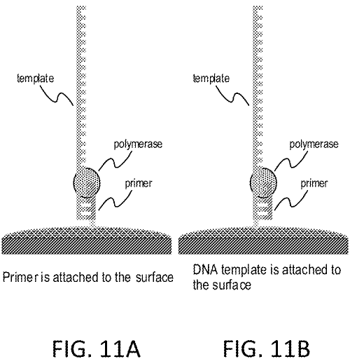

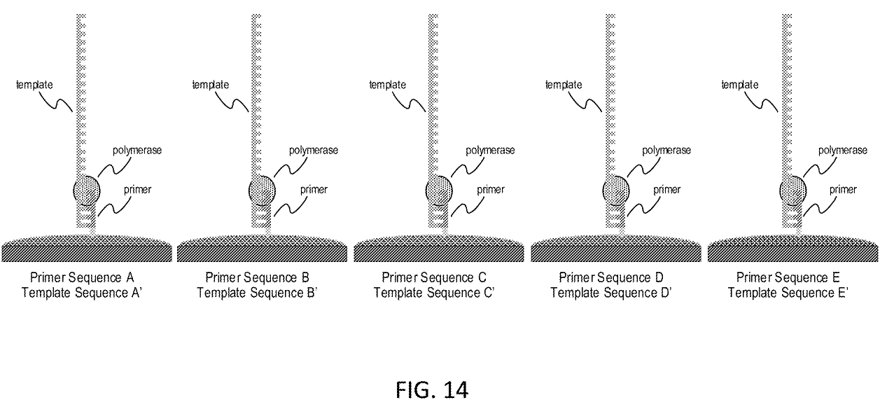

Exemplary embodiments for providing the components of invention sequencing mixture in a confinement area include among numerous other configurations, those that are shown in FIGS. 10-14. For example, in one embodiment, each target nucleic acid template is bound to the surface of an individual respective signal detector. In one embodiment, the nucleic acid template can be directly bound or attached to the surface or solid substrate using numerous methods well-known in the art, such as for example, via a thiol bond to a gold surface, or the like (FIG. 11B). In other embodiments, DNA templates can be directly bound or attached to a respective surface, via silanes, an NHS ester, or the like. In other embodiments, primers for sequencing can be bound to the surface of an individual respective signal detector (FIG. 11A). As set forth herein, each attachment can be on a surface of a individual signal detector. Exemplary signal detectors have been described herein, and can be pixels of a CCD, CMOS sensor, or they can be a photodetector, or photomultiplier forming an array, or the like.

Where the substrates comprise arrays of optical confinements, the arrays may comprise a single row or a plurality of rows of optical confinement on the surface of a substrate, where when a plurality of lanes are present, the number of lanes will usually be at least 2, more commonly more than 10, and more commonly more than 100. The subject array of optical confinements may align horizontally or diagonally long the x-axis or the y-axis of the substrate. The individual confinements can be arrayed in any format across or over the surface of the substrate, such as in rows and columns so as to form a grid, or to form a circular, elliptical, oval, conical, rectangular, triangular, or polyhedral pattern. To minimize the nearest-neighbor distance between adjacent optical confinements, a hexagonal array is sometimes preferred.

The array of optical confinements may be incorporated into a structure that provides for ease of analysis, high throughput, or other advantages, such as in a microtiter plate and the like. Such setup is also referred to herein as an “array of arrays.” For example, the subject arrays can be incorporated into another array such as microtiter plate wherein each micro well of the plate contains a subject array of optical confinements.

In accordance with the invention, arrays of confinements (e.g., reaction cells, micro-droplets, and the like) are provided in arrays of more than 100, more than 1000, more than 10,000, more than 100,000, or more than 1,000,000 separate reaction cells (such as a micro-droplet or the like) on a single substrate. In addition, the reaction cell arrays are typically comprised in a relatively high density on the surface of the substrate. Such high density typically includes reaction cells present at a density of greater than 10 reaction cells per mm², preferably, greater than 100 reaction cells per mm² of substrate surface area, and more preferably, greater than 500 or even 1000 reaction cells per mm² and in many cases up to or greater than 100,000 reaction cells per mm mm². Although in many cases, the reaction cells in the array are spaced in a regular pattern, e.g., in 2, 5, 10, 25, 50 or 100 or more rows and/or columns of regularly spaced reaction cells in a given array, in certain preferred cases, there are advantages to providing the organization of reaction cells in an array deviating from a standard row and/or column format. In preferred aspects, the substrates include as the particular reaction cell micro-droplets as the optical confinements to define the discrete single molecule sequencing reaction regions on the substrate.

The overall size of the array of optical confinements can generally range from a few nanometers to a few millimeters in thickness, and from a few millimeters to 50 centimeters in width and/or length. Arrays may have an overall size of about few hundred microns to a few millimeters in thickness and may have any width or length depending on the number of optical confinements desired.

The spacing between the individual confinements can be adjusted to support the particular application in which the subject array is to be employed. For instance, if the intended application requires a dark-field illumination of the array without or with a low level of diffractive scattering of incident wavelength from the optical confinements, then the individual confinements may be placed close to each other relative to the incident wavelength.

The individual confinement in the array can provide an effective observation volume less than about 1000 zeptoliters, less than about 900, less than about 200, less than about 80, less than about 10 zeptoliters. Where desired, an effective observation volume less than 1 zeptoliter can be provided. In a preferred aspect, the individual confinement yields an effective observation volume that permits resolution of individual molecules, such as enzymes, present at or near a physiologically relevant concentration. The physiologically relevant concentrations for many biochemical reactions range from micro-molar to millimolar because most of the enzymes have their Michaelis constants in these ranges. Accordingly, preferred array of optical confinements has an effective observation volume for detecting individual molecules present at a concentration higher than about 1 micromolar (uM), or more preferably higher than 50 uM, or even higher than 100 uM. In particular embodiments, typical microdroplet sizes range from 10 micrometers to 200 micrometers, and thus typical microdroplet volumes are around 5 picoliters to 20 nanoliters.

In the context of chemical or biochemical analyses within optical confinements, it is generally desirable to ensure that the reactions of interest are taking place within the optically interrogated portions of the confinement, at a minimum, and preferably such that only the reactions of a single molecule polymerase sequencing reaction is occurring within an interrogated portion of an individual confinement (e.g., within a micro-droplet, or the like). A number of methods well-known in the art may generally be used to provide individual molecules within the observation volume. A variety of these are described in U.S. Pat. 7,763,423, incorporated herein by reference in its entirety for all purposes, which describes, inter alia, modified surfaces that are designed to immobilize individual molecules to the surface at a desired density, such that approximately one, two, three or some other select number of molecules would be expected to fall within a given observation volume. Typically, such methods utilize dilution techniques to provide relatively low densities of coupling groups on a surface, either through dilution of such groups on the surface or dilution of intermediate or final coupling groups that interact with the molecules of interest, or combinations of these. Also contemplated herein is the use of these dilution techniques for providing one, two, three or some other select number of single molecule sequencing reactions to fall within a given observation volume without being immobilized to a surface, such as would occur in the micro-droplet reaction cell contemplated herein for optical confinement. In a particular embodiment, the dilution techniques are utilized to provide a single molecule sequencing reaction in a micro-droplet for use in the invention sequencing method.

The systems and methods of the inventions can result in improved sequence determination and improved base calling by monitoring the signal from the luminescent-substrate-attached-leaving-groups of the nucleotide-conjugate-analogs after undergoing the 2 enzyme pol-luciferase reaction set forth herein using systems well-known in the art. In general, signal data is received by the processor. The information received by the processor can come directly from the detection optics, or the signal from the detection optics can be treated by other processors before being received by the processor. A number of initial calibration operations may be applied. Some of these initial calibration steps may be performed just once at the beginning of a run or on a more continuous basis during the run. These initial calibration steps can include such things as centroid determination, alignment, gridding, drift correction, initial background subtraction, noise parameter adjustment, frame-rate adjustment, etc. Some of these initial calibration steps, such as binning, may involve communication from the processor back to the detector/camera, as discussed further below.

Generally, some type of spectral trace determination, spectral trace extraction, or spectral filters are applied to the initial signal data. Some or all of these filtration steps may optionally be carried out at a later point in the process, e.g., after the pulse identification step. The spectral trace extraction/spectral filters may include a number of noise reduction and other filters as is well-known in the art. Spectral trace determination is performed at this stage for many of the example systems discussed herein because the initial signal data received are the light levels, or photon counts, captured by a series of adjacent pixel detectors. For example, in one example system, pixels (or intensity levels) from positions are captured for an individual wave-guide at each frame. Light of different frequencies or spectrum will fall on more than one of the positions and there is generally some overlap and possibly substantial overlap. According to specific embodiments of the invention, spectral trace extraction may be performed using various type of analyses, as discussed below, that provide the highest signal-to-noise ratio for each spectral trace.

As an alternative to a spectral trace determination, methods of the invention may also analyze a single signal derived from the intensity levels at the multiple pixel positions (this may be referred to as a summed spectral signal or a gray-scale spectral signal or an intensity level signal). In many situations, it has been found that spectral extraction, however, provides better SNR (signal to noise ratio) and therefore pulse detection when extracted spectral traces are analyzed for pulses somewhat separately. In further embodiments, a method according to the invention may analyze the multiple captured pixel data using a statistical model such as a Hidden Markov Model. In the invention sequencing methods and systems provided herein, determining multiple (e.g., four) spectral traces from the initial signal data is a preferred method.

Whether the signal from the luminescent-substrate-attached-leaving-groups (e.g., PPi-Cl or PPi-FMNH2) can be categorized as a significant signal pulse or event is determined. In some example systems, because of the small number of photons available for detection and because of the speed of detection, various statistical analysis techniques may be performed in determining whether a significant pulse has been detected.

If the signal is identified as a significant pulse or signal event, a further optional spectral profile comparison may be performed to verify the spectral assignment. This spectral profile comparison is optional in embodiments where spectral traces are determined prior to or during pulse identification. Once a color is assigned to a given incorporation signal (e.g., a particular nucleotide-conjugate-analog; dNTP-Cl or dNTP-FMNH2), that assignment is used to call either the respective base incorporated, or its complement in the template sequence. In order to make this determination, the signals coming from the channel corresponding to the respective luminescent-substrate-attached-leaving-groups (e.g., PPi-Luminescent-Substrate) are used to assess whether a pulse from a nucleotide label corresponds to an incorporation event. The compilation of called bases is then subjected to additional processing to provide linear sequence information, e.g., the successive sequence of nucleotides in the template sequence, assemble sequence fragments into longer contigs, or the like.

As noted above, the signal data is input into the processing system, e.g., an appropriately programmed computer or other processor. Signal data may input directly from a detection system, e.g., for real time signal processing, or it may be input from a signal data storage file or database. In some cases, e.g., where one is seeking immediate feedback on the performance of the detection system, adjusting detection or other experimental parameters, real-time signal processing will be employed. In some embodiments, signal data is stored from the detection system in an appropriate file or database and is subject to processing in post reaction or non-real time fashion.

The signal data used in conjunction with the present invention may be in a variety of forms. For example, the data may be numerical data representing intensity values for optical signals received at a given detector or detection point of an array based detector. Signal data may comprise image data from an imaging detector, such as a CCD, EMCCD, ICCD or CMOS sensor. In particular embodiments, for detecting low numbers of photons from single molecules, the use of a photomultiplier tube (PMT) and/or a photon counter unit is contemplated for use in the invention methods. In either event, signal data used according to specific embodiments of the invention generally include both intensity level information and spectral information. In the context of separate detector elements, such spectral information will generally include identification of the location or position of the detector portion (e.g., a pixel) upon which an intensity is detected. In the context of image data, the spectral image data will typically be the data derived from the image data that correlates with the calibrated spectral image data for the imaging system and detector when the system includes spectral resolution of overall signals. The spectral data may be obtained from the image data that is extracted from the detector, or alternatively, the derivation of spectral data may occur on the detector such that spectral data will be extracted from the detector.

For the sequencing methods described above, there may be a certain amount of optical signal that is detected by the detection system that is not the result of a signal from an incorporation event. Such signal will represent “noise” in the system, and may derive from a number of sources that may be internal to the monitored reaction, internal to the detection system and/or external to all of the above. The practice of the present invention advantageously reduces these overall sources of noise typically present in prior art methods. Examples of prior art noise internal to the reaction that is advantageously reduced in accordance with the present invention includes, e.g.: presence of optical or light emitting events that are not associated with a detection event, e.g., light emission associated with unincorporated bases in diffused in solution, bases associated with the complex but not incorporated; presence of multiple complexes in an individual observation volume or region; non-specific adsorption of nucleotides to a substrate or enzyme complex within an observation volume; contaminated nucleotide analogs; spectrally shifting dye components, e.g., as a result of reaction conditions; and the like. The controlled use of luminescent signal detection and information from the luminescent-substrate on the luminescent-substrate-attached-leaving-groups of the respective dNTP that undergoes a discreet, limited-period Polymerase-Luciferase reaction prior to the incorporation of the next nucleotide-conjugate-analog advantageously provides a way of reducing or eliminating sources of noise, thereby improving the signal to noise of the system, and improving the quality of the base calls and associated sequence determination.

Sources of noise internal to the detection system, but outside of the reaction mixture can include, e.g., reflected excitation radiation that bleeds through the filtering optics; scattered excitation or luminescent radiation from the substrate or any of the optical components; spatial cross-talk of adjacent signal sources; read noise from the detector, e.g., CCDs, gain register noise, e.g., for EMCCD cameras, and the like. Other system derived noise contributions can come from data processing issues, such as background correction errors, focus drift errors, autofocus errors, pulse frequency resolution, alignment errors, and the like. Still other noise contributions can derive from sources outside of the overall system, including ambient light interference, dust, and the like.

These noise components contribute to the background photons underlying any signal pulses that may be associated with an incorporation event. As such, the noise level will typically form the limit against which any signal pulses may be determined to be statistically significant.

Identification of noise contribution to overall signal data may be carried out by a number of methods well-known in the art, including, for example, signal monitoring in the absence of the reaction of interest, where any signal data is determined to be irrelevant. Alternatively, and preferably, a baseline signal is estimated and subtracted from the signal data that is produced by the system, so that the noise measurement is made upon and contemporaneously with the measurements on the reaction of interest. Generation and application of the baseline may be carried out by a number of means, which are described in greater detail below.

In accordance with the present invention, signal processing methods distinguish between noise, as broadly applied to all non-significant pulse-based signal events, and significant signal pulses that may, with a reasonable degree of confidence, be considered to be associated with, and thus can be tentatively identified as, an incorporation event. In the context of the present invention, a signal event is first classified as to whether it constitutes a significant signal pulse based upon whether such signal event meets any of a number of different pulse criteria. Once identified or classified as a significant pulse, the signal pulse may be further assessed to determine whether the signal pulse constitutes an incorporation event and may be called as a particular incorporated base. As will be appreciated, the basis for calling a particular signal event as a significant pulse, and ultimately as an incorporation event, will be subject to a certain amount of error, based upon a variety of parameters as generally set forth herein. As such, it will be appreciated that the aspects of the invention that involve classification of signal data as a pulse, and ultimately as an incorporation event or an identified base, are subject to the same or similar errors, and such nomenclature is used for purposes of discussion and as an indication that it is expected with a certain degree of confidence that the base called is the correct base in the sequence, and not as an indication of absolute certainty that the base called is actually the base in a given position in a given sequence.

One such signal pulse criterion is the ratio of the signals associated with the signal event in question to the level of all background noise (“signal to noise ratio” or “SNR”), which provides a measure of the confidence or statistical significance with which one can classify a signal event as a significant signal pulse. In distinguishing a significant pulse signal from systematic or other noise components, the signal generally must exceed a signal threshold level in one or more of a number of metrics, including for example, signal intensity, signal duration, temporal signal pulse shape, pulse spacing, and pulse spectral characteristics.

By way of a simplified example, signal data may be input into the processing system. If the signal data exceeds a signal threshold value in one or more of signal intensity and signal duration, it may be deemed a significant pulse signal. Similarly, if additional metrics are employed as thresholds, the signal may be compared against such metrics in identifying a particular signal event as a significant pulse. As will be appreciated, this comparison will typically involve at least one of the foregoing metrics, and preferably at least two such thresholds, and in many cases three or all four of the foregoing thresholds in identifying significant pulses.

Signal threshold values, whether in terms of signal intensity, signal duration, pulse shape, spacing or pulse spectral characteristics, or a combination of these, will generally be determined based upon expected signal profiles from prior experimental data, although in some cases, such thresholds may be identified from a percentage of overall signal data, where statistical evaluation indicates that such thresholding is appropriate. In particular, in some cases, a threshold signal intensity and/or signal duration may be set to exclude all but a certain fraction or percentage of the overall signal data, allowing a real-time setting of a threshold. Again, however, identification of the threshold level, in terms of percentage or absolute signal values, will generally correlate with previous experimental results. In alternative aspects, the signal thresholds may be determined in the context of a given evaluation. In particular, for example, a pulse intensity threshold may be based upon an absolute signal intensity, but such threshold would not take into account variations in signal background levels, e.g., through reagent diffusion, that might impact the threshold used, particularly in cases where the signal is relatively weak compared to the background level. As such, in certain aspects, the methods of the invention determine the background luminescence of the particular reaction in question, which is relatively small because the contribution of freely diffusing luminescent-substrates or nucleotide-conjugate-analogs into a micro-droplet is minimal or non-existent, and sets the signal threshold above that actual background by the desired level, e.g., as a ratio of pulse intensity to background luminescent-substrate diffusion, or by statistical methods, e.g., 5 sigma, or the like. By correcting for the actual reaction background, such as the minimal luminescent-substrate diffusion background, the threshold is automatically calibrated against influences of variations in dye concentration, laser power, or the like. By reaction background is meant the level of background signal specifically associated with the reaction of interest and that would be expected to vary depending upon reaction conditions, as opposed to systemic contributions to background, e.g., autoluminescence of system or substrate components, laser bleedthrough, or the like.

In particularly preferred aspects that rely upon real-time detection of incorporation events, identification of a significant signal pulse may rely upon a signal profile that traverses thresholds in both signal intensity and signal duration. For example, when a signal is detected that crosses a lower intensity threshold in an increasing direction, ensuing signal data from the same set of detection elements, e.g., pixels, are monitored until the signal intensity crosses the same or a different intensity threshold in the decreasing direction. Once a peak of appropriate intensity is detected, the duration of the period during which it exceeded the intensity threshold or thresholds is compared against a duration threshold. Where a peak comprises a sufficiently intense signal of sufficient duration, it is called as a significant signal pulse.

In addition to, or as an alternative to using the intensity and duration thresholds, pulse classification may employ a number of other signal parameters in classifying pulses as significant. Such signal parameters include, e.g., pulse shape, spectral profile of the signal, e.g., pulse spectral centroid, pulse height, pulse diffusion ratio, pulse spacing, total signal levels, and the like.

Either following or prior to identification of a significant signal pulse, signal data may be correlated to a particular signal type. In the context of the optical detection schemes used in conjunction with the invention, this typically denotes a particular spectral profile of the signal giving rise to the signal data. In particular, the optical detection systems used in conjunction with the methods and processes of the invention are generally configured to receive optical signals that have distinguishable spectral profiles, where each spectrally distinguishable signal profile may generally be correlated to a different reaction event. In the case of nucleic acid sequencing, for example, each spectrally distinguishable signal may be correlated or indicative of a specific nucleotide incorporated or present at a given position of a nucleic acid sequence. Consequently, the detection systems include optical trains that receive such signals and separate the signals based upon their spectra. The different signals are then directed to different detectors, to different locations on a single array based detector, or are differentially imaged upon the same imaging detector (See, e.g., U.S. Pat. 7,805,081, which is incorporated herein by reference in its entirety for all purposes).

In the case of systems that employ different detectors for different signal spectra, assignment of a signal type (for ease of discussion, referred to hereafter as “color classification,” “wave length” or “spectral classification”) to a given signal is a matter of correlating the signal pulse with the detector from which the data derived. In particular, where each separated signal component is detected by a discrete detector, a signal’s detection by that detector is indicative of the signal classifying as the requisite color.

In preferred aspects, however, the detection systems used in conjunction with the invention utilize an imaging detector upon which all or at least several of the different spectral components of the overall signal are imaged in a manner that allows distinction between different spectral components. Thus, multiple signal components are directed to the same overall detector, but may be incident upon wholly or partly different regions of the detector, e.g., imaged upon different sets of pixels in an imaging detector, and give rise to distinguishable spectral images (and associated image data). As used herein, spectra or spectral image generally indicates a pixel image or frame (optionally data reduced to one dimension) that has multiple intensities caused by the spectral spread of an optical signal received from a reaction location.

In its simplest form, it will be understood that assignment of color to a signal event incident upon a group of contiguous detection elements or pixels in the detector would be accomplished in a similar fashion as that set forth for separate detectors. In particular, the position of the group of pixels upon which the signal was imaged, and from which the signal data is derived, is indicative of the color of the signal component. In particularly preferred aspects, however, spatial separation of the signal components may not be perfect, such that signals of differing colors are imaged on overlapping sets of pixels. As such, signal identification will generally be based upon the aggregate identity of multiple pixels (or overall image of the signal component) upon which a signal was incident.

Once a particular signal is identified as a significant pulse and is assigned a particular spectrum, the spectrally assigned pulse may be further assessed to determine whether the pulse can be called an incorporation event and, as a result, call the base incorporated in the nascent strand, or its complement in the template sequence. Signals from the luminescent-substrate-attached-leaving-groups (e.g., PPi-C1, PPi-FMNH2, or the like) are used to identify which base should be called. As set forth above, in one embodiment, by using the invention 2 enzyme polymerase-Luciferase reaction system, a set of characteristic signals are produced which can be correlated with high confidence to an incorporation event.

In addition, calling of bases from color assigned pulse data will typically employ tests that again identify the confidence level with which a base is called. Typically, such tests will take into account the data environment in which a signal was received, including a number of the same data parameters used in identifying significant pulses. For example, such tests may include considerations of background signal levels, adjacent pulse signal parameters (spacing, intensity, duration, etc.), spectral image resolution, and a variety of other parameters. Such data may be used to assign a score to a given base call for a color assigned signal pulse, where such scores are correlative of a probability that the base called is incorrect, e.g., 1 in 100 (99% accurate), 1 in 1000 (99.9% accurate), 1 in 10,000 (99.99% accurate), 1 in 100,000 (99.999% accurate), or even greater. Similar to PHRED or similar type scoring for chromatographically derived sequence data, such scores may be used to provide an indication of accuracy for sequencing data and/or filter out sequence information of insufficient accuracy.

Once a base is called with sufficient accuracy, subsequent bases called in the same sequencing run, and in the same primer extension reaction, may then be appended to each previously called base to provide a sequence of bases in the overall sequence of the template or nascent strand. Iterative processing and further data processing can be used to fill in any blanks, correct any erroneously called bases, or the like for a given sequence.

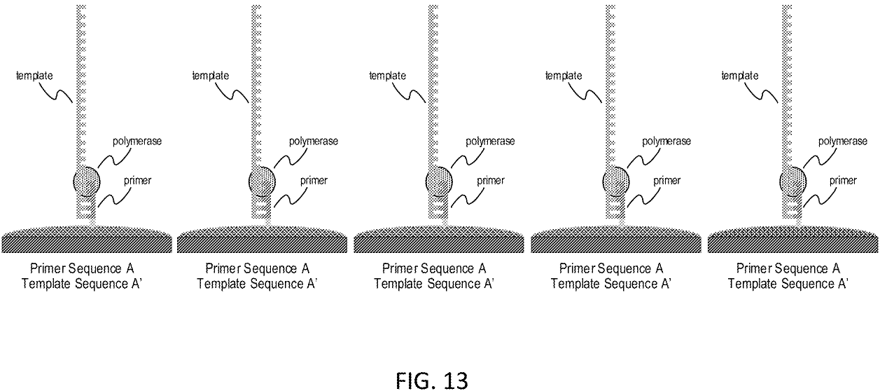

Analysis of sequencing-by-incorporation-reactions on an array of reaction locations according to specific embodiments of the invention can be conducted as illustrated graphically in FIG. 13 of US Pat. 9,447,464, incorporated by reference in its entirety for all purposes). For example, data captured by a camera is represented as a movie, which is also a time sequence of spectra. Spectral calibration templates are used to extract traces from the spectra. Pulses identified in the traces are then used to return to the spectra data and from that data produce a temporally averaged pulse spectrum for each pulse, such pulse spectra will include spectra for events relating to enzyme conformational changes. The spectral calibration templates are then also used to classify pulse spectrum to a particular base. Base classifications and pulse and trace metrics are then stored or passed to other logic for further analysis. The downstream analysis will include using the information from enzyme conformational changes to assist in the determination of incorporation events for base calling. Further base calling and sequence determination methods for use in the invention can include those described in, for example, U.S. 8,182,993, which is incorporated herein by reference in its entirety for all purposes.

An advantage of the invention single molecule sequencing methods that permit the use of polymerase in an environment that is more optimized for polymerase, is the very low error rate achieved per sequencing run; or in other words the substantially high level of sequence accuracy obtained per sequencing run. For example, natural polymerase makes 1 error per 100 million bases; and this is contemplated herein as target error rate for the invention LASH sequencing methods provided herein. Also in accordance with the present invention that uses a plurality of polymerases per target nucleic template, the error rate is independent of read length; therefore, the error rate can be improved by the selection of a higher fidelity polymerase and as a result require less coverage; and still can achieve very long read length by using a plurality of polymerases. Error rates achieved by polymerases used in the invention methods, per run before coverage is considered, are contemplated to be in the range selected from: 1%-30%, 1%-20%, 1%-10%, 1%-5%, 1%-3%, 1%-2%, 0.000001% - 1%, 0.00001%-1%, 0.0001% - 1%, 0.001%-1%, 0.01%-1%, 0.000001%-0.00001%, 0.000001%-0.0001%, 0.000001%-0.001%.

This advantage reduces the overall coverage required for obtaining an accurate sequence as defined by industry standards, which correspondingly reduces the overall cost of obtaining the nucleotide sequence. As used herein, coverage refers the number of sequencing runs required to obtain an accurate sequence for a particular target nucleic acid sequence within industry standards.

### EXAMPLES

### Example 1 - Luminescence-Based Single Molecule Sequencing

Prior to undergoing a single molecule sequencing reaction, the respective luminescence substrates are attached to the terminal phosphate of its corresponding dNTP for each of dATP, dTTP, dGTP and dCTP. There is a different luminescent-substrate for each dNTP base (A, T, G, C) (FIG. 1A & FIG. 1B). During the single molecule sequencing reaction, upon interaction with the DNA polymerase, while the DNA polymerase binds the dNTP nucleotide-conjugate-analog to the complementary template strand, it cleaves off and releases a pyrophosphate that includes the luminescent-substrate attached thereto (PPi-C1, FIG. 2B and PPi-FMNH2, FIG. 3B).

Once released, the labeled pyrophosphate (PPᵢ-C1; PPi-FMNH2) is used to bind to a luciferase that, as a result of the enzymatic catalysis, produces luminescence for a discreet and limited time (FIG. 2C and FIG. 3C). This results in a detectable luminescence emission during the discreet and limited period (lifetime) of the bioluminescence, which spectra of light emission corresponds to the respective dNTP incorporated into the template strand. Accordingly, as a result of dNTP interacting with the DNA polymerase, luminescence light is generated by the luminescence reaction produced by the luminescence-enzyme and luminescence-substrate, generating a luminescence signal corresponding to the wavelength selected for the particular dNTP. The respective luminescent light is the detected prior to the light vanishing after a discreet and limited period of time, such as in one embodiment, before the addition of the next dNTP.

This dNTP incorporation process is repeated until the desired nucleic acid read-length has been achieved.

While the present embodiments have been particularly shown and described with reference to example embodiments herein, it will be understood by those of ordinary skill in the art that various changes in form and details may be made therein without departing from the spirit and scope of the present embodiments as defined by the following claims. Those skilled in the art will recognize, or be able to ascertain using no more than routine experimentation, numerous equivalents to the specific procedures described herein. Such equivalents are considered to be within the scope of the present invention and are covered by the following claims. The contents of all non-patent literature publications, patents, and patent applications cited throughout this application are hereby incorporated by reference in their entirety for all purposes. The appropriate components, processes, and methods of those patents, applications and other documents may be selected for the present invention and embodiments thereof.

What is claimed is:

1. A method for sequencing a nucleic acid template comprising:
providing a sequencing mixture comprising (i) a polymerase enzyme, (ii) a luminescence enzyme, (iii) a template nucleic acid and primer, and (iv) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has a different luminescent-substrate attached thereto, wherein the luminescent-substrate-attached-leaving-group is cleaved upon polymerase-dependent binding of a respective nucleotide-conjugate-analog to the template strand;
carrying out nucleic acid synthesis such that a plurality of nucleotide-conjugate-analogs are added sequentially to the template whereby: a) a nucleotide-conjugate-analog associates with the polymerase, b) the nucleotide-conjugate-analog is incorporated on the template strand by the polymerase when the luminescent-substrate-attached-leaving-group on that nucleotide-conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce nucleotide-specific-luminescence for a limited period of time; and
detecting nucleotide-specific-luminescence signal (light) while nucleic acid synthesis is occurring, and using nucleotide-specific-luminescence signal detected during each discreet luminescence period to determine a sequence of the template nucleic acid.

2. The method of claim 1, wherein the luminescent-substrate is selected from the group consisting of: colentarazine or an analog thereof; FMNH2 or an analog thereof; luminol, isoluminol, acridinium, dioxetanes, peroxyozalic, and their derivatives thereof.

3. The method of claims 1-2, wherein each base of a nucleotide is labeled with a unique luminescent-substrate relative to other bases.

4. The method of claims 1-3, wherein the luminescence-enzyme is a luciferase or photoprotein.

5. The method of claims 1-4, wherein the luciferase is selected from the group consisting of: Renilla Luciferase, Gaussia Luciferase, Vibrio harveyi luciferase, Vibrio fischeri luciferase, Photobacterium fischeri luciferase, Photobacterium phosphoreum luciferase, P. leiognathi luciferase, and P. luminescens luciferase.

6. The method of claims 1-4, wherein the photoprotein is selected from the group consisting of: aequorin and obelin,.

7. The method of claims 1-6, wherein the polymerase enzyme is DNA polymerase.

8. The method of claims 1-7, wherein types of nucleotide-conjugate-analogs comprise a nucleotide selected from the group consisting of: dATP, dTTP, dGTP, dCTP, dUTP, dGTPαS, dCTPαS, dTTPαS and dATPαS.

9. The method of claims 1-8, wherein a plurality of polymerase enzymes are used.

10. A method of sequencing a template nucleic acid, comprising:
providing a sequencing mixture comprising: a target template nucleic acid, a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has a different luminescent-substrate attached thereto, a luminescence-enzyme, and plurality of polymerase enzymes;
carrying out nucleic acid synthesis such that a plurality of nucleotide-conjugate-analogs are added sequentially to the template; and
detecting a respective nucleotide-conjugate-analog while nucleic acid synthesis is occurring, to determine a sequence of the template nucleic acid.

11. A method for detecting the presence of a target nucleic acid sequence in a sample comprising:
providing an elongation mixture comprising (i) a polymerase enzyme, (ii) a luminescence enzyme, (iii) a template nucleic acid sample, (iv) a primer-probe that hybridizes to (e.g., that is complementary to) a particular target nucleic acid sequence, and (v) a polymerase-luminescence reagent solution having the components for carrying out template directed synthesis of a growing nucleic acid strand, wherein said reagent solution includes a plurality of types of nucleotide-conjugate-analogs, each having a luminescent-substrate attached thereto; wherein each type of nucleotide-conjugate-analog has a luminescent-substrate-attached-leaving-group that is cleavable by the polymerase, and each type of nucleotide-conjugate-analog has the same, or different, luminescent-substrate attached thereto, wherein the luminescent-substrate-attached-leaving-group is cleaved upon polymerase-dependent binding of a respective nucleotide-conjugate-analog to the template strand;
carrying out nucleic acid elongation synthesis such that a plurality of nucleotide-conjugate-analogs are added sequentially to the template if the primer-probe hybridizes to the target nucleic acid sequence, whereby: a) a nucleotide-conjugate-analog associates with the polymerase, b) the nucleotide-conjugate-analog is incorporated on the template strand by the polymerase when the luminescent-substrate-attached-leaving-group on that nucleotide-conjugate-analog is cleaved by the polymerase, wherein the luminescent-substrate-attached-leaving-group is combined with the luminescence-enzyme in a luminescence reaction, wherein the luminescence-substrate is catalyzed by the luminescence-enzyme to produce luminescence; and
detecting light from the luminescence while nucleic acid synthesis is occurring, whereby detection of light indicates the presence of the particular target nucleic acid sequence.

12. The method of claim 11, wherein the amount of target nucleic acid is quantified.

13. The method of claim 11, wherein the amount of target nucleic acid is quantified based on the intensity of the luminescence.

14. The method of claims 11-13, wherein each type of nucleotide-conjugate-analog has the same luminescent-substrate attached thereto.

15. The method of claims 1-14, wherein a plurality of polymerase enzymes are used.

16. The method of claims 1-15, wherein a plurality of polymerase enzymes are use in an amount selected from the group consisting of at least: 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 10000, 20000, 30000, 40000, 50000, 60000, 70000, 80000, 90000, 100000, 200000, 300000, 400000, 500000, 600000, 700000, 800000, 900000, and at least 1000000 polymerase enzymes.

17. The method of claims 1-16, wherein a plurality of polymerase enzymes are use in a ratio of polymerase to template is selected from the group consisting of at least 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 10000:1, 20000:1, 30000:1, 40000:1, 50000:1, 60000:1, 70000:1, 80000:1, 90000:1, 100000:1, 200000:1, 300000:1, 400000:1, 500000:1, 600000:1, 700000:1, 800000:1, 900000:1, and at least 1000000:1.

18. A luminescent-substrate-nucleotide-conjugate-analog, comprising a deoxyribonucleotide (dNTP), or analog thereof; and a luminescent-substrate attached thereto.

19. The luminescent-substrate-nucleotide-conjugate-analog of claim 18,, wherein the nucleotide (dNTP) within the luminescent-substrate-nucleotide-conjugate-analogs are modified nucleotide analogs.

20. The luminescent-substrate-nucleotide-conjugate-analog of claim 18, wherein the dNTP is selected from the group consisting of: dATP, dTTP, dGTP, dCTP and dUTP, dATPαS, dGTPαS, dCTPαS, dTTPαS and dUTPαS.

21. The luminescent-substrate-nucleotide-conjugate-analog of claim 18 wherein the nucleotide-conjugate-analog is capable of being a substrate for the polymerase and for the selective cleaving activity.

22. The luminescent-substrate-nucleotide-conjugate-analog of claim 18, wherein the nucleotide-conjugate-analog is a nucleoside polyphosphate having three or more phosphates in its polyphosphate chain with a luminescent substrate attached to the portion of the polyphosphate chain that is cleaved upon incorporation into a growing template directed strand.

23. The luminescent-substrate-nucleotide-conjugate-analog of claim 22, wherein the polyphosphate is a pure polyphosphate (—O—PO3—), a pyrophosphate (PPi), or polyphosphate having substitutions therein.

24. The luminescent-substrate-nucleotide-conjugate-analog of claim 18, wherein the luminescent-substrate is selected from the group consisting of: colentarazine or an analog thereof; FMNH2 or an analog thereof; luminol, isoluminol, acridinium, dioxetanes, peroxyozalic, and their derivatives thereof.

25. The luminescent-substrate-nucleotide-conjugate-analog of claim 18, wherein the luminescent-substrate is attached to a terminal phosphate.

26. The luminescent-substrate-nucleotide-conjugate-analog of claim 25, wherein when the PPi luminescent-substrate-attached-leaving-group is generated by the polymerase when the luminescent-substrate nucleotide-conjugate is incorporated into the template strand, the luminescent-substrate-attached-pyrophosphate or luminescent-substrate-attached-leaving-group is able to be combined with the respective luciferase.

27. The luminescent-substrate-nucleotide-conjugate-analog of claim 26, wherein the PPi luminescent-substrate-attached-leaving-group is selected from PPi-LS, PPi-C; PPi-FMNH2.

28. The luminescent-substrate-nucleotide-conjugate-analog of claim 18, wherein the nucleotide-conjugate-analog has a unique luminescent signal.

29. The luminescent-substrate-nucleotide-conjugate-analog of claim 28, wherein the luminescent signal is a wavelength selected from the range 250 nm - 750 nm.

30. The luminescent-substrate-nucleotide-conjugate-analog of claim 28, wherein the luminescent signal is a wavelength selected from the group consisting of: 411, 417, 428, 440, 484, and 509 nm.

31. A chain-elongation set of nucleotide-conjugate-analogs comprising at least 4 distinct a deoxyribonucleotides (dNTPs), such that the chain-elongation set can be incorporated into template directed synthesis of a growing nucleic acid strand.

32. The chain-elongation set of nucleotide-conjugate-analogs of claim 31, wherein each respective dNTP, or analog thereof, is modified using a different, unique luminescent substrate relative to the other dNTPs, such that each time a polymerase incorporates a modified deoxyribonuleoside triphosphate (dNTP) nucleotide-conjugate-analog to the strand complementary to the template DNA, a luminescent signal specific to the respective nucleotide attached is generated.

33. The chain-elongation set of nucleotide-conjugate-analogs of claim 31, wherein if both modified dTTP and dUTP analogs are used in the reaction, they can each have the same luminescent substrate attached thereto producing the same wavelength signal; or each can have a discreet luminescent substrate attached thereto.

34. The chain-elongation set of nucleotide-conjugate-analogs of claim 31, wherein the dNTP is selected from the group consisting of: dATP, dTTP, dGTP, dCTP and dUTP, dATPαS, dGTPαS, dCTPαS, dTTPαS and dUTPαS.

35. The chain-elongation set of nucleotide-conjugate-analogs of claim 31, wherein luminescent-substrate is selected from the group consisting of: colentarazine or an analog thereof; FMNH2 or an analog thereof; luminol, isoluminol, acridinium, dioxetanes, peroxyozalic, and their derivatives thereof.

36. The chain-elongation set of nucleotide-conjugate-analogs of claim 31, selected from Coelenterazine-dNTP Conjugate 1 (Fig. 7); Coelentarazine-dNTP Conjugate 2 (Fig. 8); or Coelentarazine-dNTP Conjugate 3 (Fig. 9).
